(19) United States
(12) Patent Application Publication
(10) Pub. No.: US 2025/0368662 A1
(43) Pub. Date: Dec. 4, 2025

(54) INHIBITORS OF INDOLEAMINE 2,3-DIOXYGENASE 1 (IDO1) AND TRYPTOPHAN 2,3-DIOXYGENASE (TDO) AND METHODS OF USING SAME

(71) Applicant: University of South Carolina, Columbia, SC (US)

(72) Inventor: Yangmei Li, Cayce, SC (US)

(73) Assignee: University of South Carolina, Columbia, SC (US)

(21) Appl. No.: 19/226,973

(22) Filed: Jun. 3, 2025

Related U.S. Application Data
(60) Provisional application No. 63/655,775, filed on Jun. 4, 2024.
(60) Provisional application No. 63/697,011, filed on Sep. 20, 2024.

(51) Int. Cl. C07D 513/04; A61K 31/429; A61K 31/4439; A61P 25/04; A61P 35/00

(52) U.S. Cl. CPC C07D 513/04; A61K 31/429; A61K 31/4439; A61P 25/04; A61P 35/00

(57) ABSTRACT

Indoleamine 2,3-dioxygenase 1 (IDO1) and/or tryptophan 2,3-dioxygenase (TDO) inhibitors and methods of using same. The inhibitors can be used in methods of inhibiting IDO1 and/or TDO, either in vivo or in vitro. The inhibitors of the invention can also be used in methods of treating IDO1- and TDO-mediated conditions, such as cancer and neuropathic pain.

### CROSS-REFERENCE TO RELATED APPLICATIONS

Priority is hereby claimed to U.S. Provisional Application 63/655,775, filed Jun. 4, 2024, and U.S. Provisional Application 63/697,011, filed Sep. 20, 2024, which are incorporated herein by reference in their entireties.

### FIELD OF THE INVENTION

The invention is directed to inhibitors of indoleamine 2,3-dioxygenase 1 (IDO1) and/or tryptophan 2,3-dioxygenase (TDO) and methods of using same, such as for inhibiting IDO1 and/or TDO or treating IDO1- and/or TDO-mediated conditions.

### BACKGROUND

Indoleamine 2,3-dioxygenase 1 (IDO1) and tryptophan 2,3-dioxygenase (TDO) are enzymes that regulate the initial rate-limiting step of the kynurenine pathway. The kynurenine pathway metabolizes over 90% Trp that is obtained via diet to produce a variety of metabolites including kynurenine, kynurenic acid, 3-hydroxykynurenine and quinolinic acid. The kynurenine pathway is involved in the modulation of many biological and physiological processes including immune response and neurotransmission. Overactive kynurenine pathway has been linked to a wide range of diseases and conditions such as cancer, depression, chronic/neuropathic pain, neurodegeneration, HIV immune dysfunction, etc. Regulating the kynurenine pathway by inhibiting the activity of IDO1 and TDO, which catalyze the initial rate-limiting step of the kynurenine pathway, is therefore a strategy for the development of medical therapeutics for these diseases. IDO1-selective inhibitors have been actively pursued for the development of cancer treatments, though several clinical trials have been halted. As Trp is also catabolized by TDO in the KP pathway, selective inhibition of IDO1 may be compensated by TDO activity. Therefore, dual inhibitors targeting both IDO1 and TDO are needed.

### SUMMARY OF THE INVENTION

One aspect of the invention is directed to compounds. The compounds in the invention can comprise a compound of Formula (I):

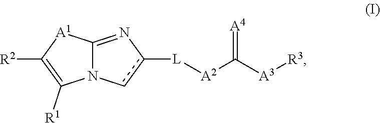

or a salt thereof, wherein:

A¹, A², A³, and A⁴ are each independently O, S, or NR⁴;
- - - represents a bond that is present or absent;
L is a bond or optionally substituted alkylene optionally containing one or more heteroatom(s);
R¹, R², R³, and R⁴ in each instance is independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

In some versions, A¹ is O. In some versions, A¹ is S. In some versions, A¹ is NR⁴.

In some versions, A² is O. In some versions, A² is S. In some versions, A² is NR⁴.

In some versions, A³ is O. In some versions, A³ is S. In some versions, A³ is NR⁴.

In some versions, A⁴ is O. In some versions, A⁴ is S. In some versions, A⁴ is NR⁴.

In some versions, - - - represents a bond that is absent. In some versions, - - - represents a bond that is present.

In some versions, L is optionally substituted alkylene containing 1-10 carbon atoms and optionally containing 1-2 heteroatom(s). In some versions, L is optionally substituted C1-C6 alkylene. In some versions, L is C1-C6 alkylene. In some versions, L is C4 alkylene.

In some versions, R¹ is not hydrogen, such that R¹ is halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

In some versions, R¹ is optionally substituted alkyl, optionally substituted cycloalkyl, optionally substituted cycloalkenyl, optionally substituted alkyloxy, optionally substituted cycloalkyloxy, optionally substituted cycloalkenyloxy, optionally substituted alkylthio, optionally substituted cycloalkylthio, optionally substituted cycloalkenylthio, optionally substituted aryl, optionally substituted aryloxy, optionally substituted arylthio, optionally substituted heteroaryl, optionally substituted heteroaryloxy, optionally substituted heteroarylthio, or an optionally substituted non-aromatic heterocyclyl.

In some versions, R¹ is Formula (II), Formula (III), Formula (IV), Formula (V), Formula (VI), Formula (VII), Formula (VIII), Formula (IX), or Formula (X):

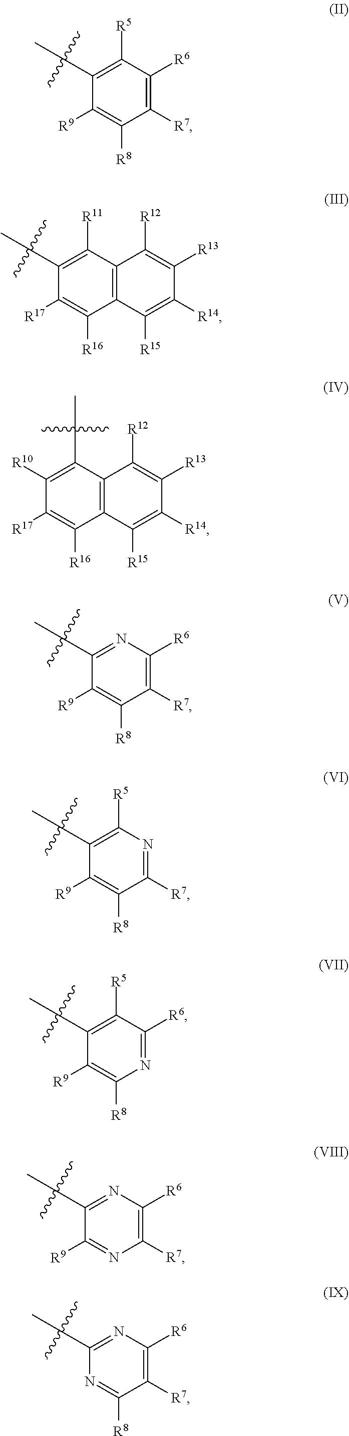

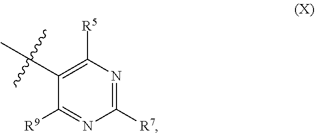

wherein R⁵—R¹⁷ of R¹ are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

In some versions, at least one of R⁵—R⁹ of Formula II, Formula V, Formula VI, Formula VII, Formula VIII, Formula IX, or Formula X of R¹ is not hydrogen. In some versions, at least one of R⁵—R⁹ of Formula II, Formula V, Formula VI, Formula VII, Formula VIII, Formula IX, or Formula X of R¹ is halogen, optionally substituted alkyloxy, or optionally substituted alkyl. In some versions, at least one of R⁵—R⁹ of Formula II, Formula V, Formula VI, Formula VII, Formula VIII, Formula IX, or Formula X of R¹ is halogen or optionally substituted alkyloxy. In some versions, at least one of R⁵—R⁹ of Formula II, Formula V, Formula VI, Formula VII, Formula VIII, Formula IX, or Formula X of R¹ is halogen. In some versions, at least one of R⁵—R⁹ of Formula II, Formula V, Formula VI, Formula VII, Formula VIII, Formula IX, or Formula X of R¹ is optionally substituted alkyloxy. In some versions, at least one of R⁵—R⁹ of Formula II, Formula V, Formula VI, Formula VII, Formula VIII, Formula IX, or Formula X of R¹ is optionally substituted alkyl.

In some versions, at least one of R⁶—R⁸ of Formula II, Formula V, Formula VI, Formula VII, Formula VIII, Formula IX, or Formula X of R¹ is not hydrogen. In some versions, at least one of R⁶—R⁸ of Formula II, Formula V, Formula VI, Formula VII, Formula VIII, Formula IX, or Formula X of R¹ is halogen, optionally substituted alkyloxy, or optionally substituted alkyl. In some versions, at least one of R⁶—R⁸ of Formula II, Formula V, Formula VI, Formula VII, Formula VIII, Formula IX, or Formula X of R¹ is halogen or optionally substituted alkyloxy. In some versions, at least one of R⁶—R⁸ of Formula II, Formula V, Formula VI, Formula VII, Formula VIII, Formula IX, or Formula X of R¹ is halogen. In some versions, at least one of R⁶—R⁸ of Formula II, Formula V, Formula VI, Formula VII, Formula VIII, Formula IX, or Formula X of R¹ is optionally substituted alkyloxy. In some versions, at least one of R⁶—R⁸ of Formula II, Formula V, Formula VI, Formula VII, Formula VIII, Formula IX, or Formula X of R¹ is optionally substituted alkyl.

In some versions, at least one of R⁶ or R⁸ of Formula II, Formula V, Formula VI, Formula VII, Formula VIII, or Formula IX of R¹ is not hydrogen. In some versions, at least one of R⁶—R⁸ of Formula II, Formula V, Formula VI, Formula VII, Formula VIII, or Formula IX of R¹ is halogen, optionally substituted alkyloxy, or optionally substituted alkyl. In some versions, at least one of R⁶—R⁸ of Formula II, Formula V, Formula VI, Formula VII, Formula VIII, or Formula IX of R¹ is halogen or optionally substituted alkyloxy. In some versions, at least one of R⁶—R⁸ of Formula II, Formula V, Formula VI, Formula VII, Formula VIII, or Formula IX of R¹ is halogen. In some versions, at least one of R⁶—R⁸ of Formula II, Formula V, Formula VI, Formula VII, Formula VIII, or Formula IX of R¹ is optionally substituted alkyloxy. In some versions, at least one of R⁶—R⁸ of Formula II, Formula V, Formula VI, Formula VII, Formula VIII, or Formula IX of R¹ is optionally substituted alkyl.

In some versions, R⁷ of Formula II, Formula V, Formula VI, Formula VIII, Formula IX, or Formula X of R¹ is not hydrogen. In some versions, R⁷ of Formula II, Formula V, Formula VI, Formula VIII, Formula IX, or Formula X of R¹ is halogen, optionally substituted alkyloxy, or optionally substituted alkyl. In some versions, R⁷ of Formula II, Formula V, Formula VI, Formula VIII, Formula IX, or Formula X of R¹ is halogen or optionally substituted alkyloxy. In some versions, R⁷ of Formula II, Formula V, Formula VI, Formula VIII, Formula IX, or Formula X of R¹ is halogen. In some versions, R⁷ of Formula II, Formula V, Formula VI, Formula VIII, Formula IX, or Formula X of R¹ is optionally substituted alkyloxy. In some versions, R⁷ of Formula II, Formula V, Formula VI, Formula VIII, Formula IX, or Formula X of R¹ is optionally substituted alkyl.

In some versions, R⁵ of Formula II, Formula VI, Formula VII, or Formula X of R¹ is hydrogen. In some versions, R⁶ of Formula II, Formula V, Formula VII, Formula VIII, or Formula IX of R¹ is hydrogen. In some versions, R⁷ of Formula II, Formula V, Formula VI, Formula VIII, Formula IX, or Formula X of R¹ is hydrogen. In some versions, R⁸ of Formula II, Formula V, Formula VI, Formula VII, or Formula IX of R¹ is hydrogen. In some versions, R⁹ of Formula II, Formula V, Formula VI, Formula VII, Formula VIII, or Formula X of R¹ is hydrogen. In some versions, R¹⁰ of Formula IV of R¹ is hydrogen. In some versions, R¹¹ of Formula III of R¹ is hydrogen. In some versions, R¹² of Formula III or Formula IV of R¹ is hydrogen. In some versions, R¹³ of Formula III or Formula IV of R¹ is hydrogen. In some versions, R¹⁴ of Formula III or Formula IV of R¹ is hydrogen. In some versions, R¹⁵ of Formula III or Formula IV of R¹ is hydrogen. In some versions, R¹⁶ of Formula III or Formula IV of R¹ is hydrogen. In some versions, R¹⁷ of Formula III or Formula IV of R¹ is hydrogen.

In some versions, R¹ is Formula II, Formula III, Formula IV, or Formula VI. In some versions, R¹ is Formula II, Formula III, or Formula IV. In some versions, R¹ is Formula II. In some versions, R¹ is Formula III. In some versions, R¹ is Formula IV. In some versions, R¹ is Formula VI.

In some versions, R¹ is: any one of Formulas II, V, VI, VII, VIII, IX, and X wherein at least one of R⁶—R⁸ is not hydrogen; or any one of Formulas III and IV. In some versions, R¹ is: any one of Formulas II, V, VI, VII, VIII, IX, and X wherein at least one of R⁶—R⁸ is halogen, optionally substituted alkyloxy, or optionally substituted alkyl; or any one of Formulas III and IV. In some versions, R¹ is: any one of Formulas II, V, VI, VII, VIII, IX, and X wherein at least one of R⁶—R⁸ is halogen or optionally substituted alkyloxy; or any one of Formulas III and IV.

In some versions, R¹ is: any one of Formulas II, V, VI, VIII, IX, and X wherein R⁷ is not hydrogen; or any one of Formulas III and IV. In some versions, R¹ is: any one of Formulas II, V, VI, VIII, IX, and X wherein R⁷ is halogen, optionally substituted alkyloxy, or optionally substituted alkyl; or any one of Formulas III and IV. In some versions, R¹ is: any one of Formulas II, V, VI, VIII, IX, and X wherein R⁷ is halogen or optionally substituted alkyloxy; or any one of Formulas III and IV.

In some versions, R² is Formula (II), Formula (III), Formula (IV), Formula (V), Formula (VI), Formula (VII), Formula (VIII), Formula (IX), or Formula (X) wherein R⁵—R¹⁷ of R² are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

In some versions, at least one of R⁵—R⁹ of Formula II, Formula V, Formula VI, Formula VII, Formula VIII, Formula IX, or Formula X of R² is not hydrogen. In some versions, at least one of R⁵—R⁹ of Formula II, Formula V, Formula VI, Formula VII, Formula VIII, Formula IX, or Formula X of R² is halogen, optionally substituted alkyloxy, or optionally substituted alkyl. In some versions, at least one of R⁵—R⁹ of Formula II, Formula V, Formula VI, Formula VII, Formula VIII, Formula IX, or Formula X of R² is halogen or optionally substituted alkyloxy. In some versions, at least one of R⁵—R⁹ of Formula II, Formula V, Formula VI, Formula VII, Formula VIII, Formula IX, or Formula X of R² is halogen. In some versions, at least one of R⁵—R⁹ of Formula II, Formula V, Formula VI, Formula VII, Formula VIII, Formula IX, or Formula X of R² is optionally substituted alkyloxy. In some versions, at least one of R⁵—R⁹ of Formula II, Formula V, Formula VI, Formula VII, Formula VIII, Formula IX, or Formula X of R² is optionally substituted alkyl.

In some versions, at least one of R⁶—R⁸ of Formula II, Formula V, Formula VI, Formula VII, Formula VIII, Formula IX, or Formula X of R² is not hydrogen. In some versions, at least one of R⁶—R⁸ of Formula II, Formula V, Formula VI, Formula VII, Formula VIII, Formula IX, or Formula X of R² is halogen, optionally substituted alkyloxy, or optionally substituted alkyl. In some versions, at least one of R⁶—R⁸ of Formula II, Formula V, Formula VI, Formula VII, Formula VIII, Formula IX, or Formula X of R² is halogen or optionally substituted alkyloxy. In some versions, at least one of R⁶—R⁸ of Formula II, Formula V, Formula VI, Formula VII, Formula VIII, Formula IX, or Formula X of R² is halogen. In some versions, at least one of R⁶—R⁸ of Formula II, Formula V, Formula VI, Formula VII, Formula VIII, Formula IX, or Formula X of R² is optionally substituted alkyloxy. In some versions, at least one of R⁶—R⁸ of Formula II, Formula V, Formula VI, Formula VII, Formula VIII, Formula IX, or Formula X of R² is optionally substituted alkyl.

In some versions, at least one of R⁶ or R⁸ of Formula II, Formula V, Formula VI, Formula VII, Formula VIII, or Formula IX of R² is not hydrogen. In some versions, at least one of R⁶—R⁸ of Formula II, Formula V, Formula VI, Formula VII, Formula VIII, or Formula IX of R² is halogen, optionally substituted alkyloxy, or optionally substituted alkyl. In some versions, at least one of R⁶—R⁸ of Formula II, Formula V, Formula VI, Formula VII, Formula VIII, or Formula IX of R² is halogen or optionally substituted alkyloxy. In some versions, at least one of R⁶—R⁸ of Formula II, Formula V, Formula VI, Formula VII, Formula VIII, or Formula IX of R² is halogen. In some versions, at least one of R⁶—R⁸ of Formula II, Formula V, Formula VI, Formula VII, Formula VIII, or Formula IX of R² is optionally substituted alkyloxy. In some versions, at least one of R⁶—R⁸ of Formula II, Formula V, Formula VI, Formula VII, Formula VIII, or Formula IX of R² is optionally substituted alkyl.

In some versions, R⁷ of Formula II, Formula V, Formula VI, Formula VIII, Formula IX, or Formula X of R² is not hydrogen. In some versions, R⁷ of Formula II, Formula V, Formula VI, Formula VIII, Formula IX, or Formula X of R² is halogen, optionally substituted alkyloxy, or optionally substituted alkyl. In some versions, R⁷ of Formula II, Formula V, Formula VI, Formula VIII, Formula IX, or Formula X of R² is halogen or optionally substituted alkyloxy. In some versions, R⁷ of Formula II, Formula V, Formula VI, Formula VIII, Formula IX, or Formula X of R² is halogen. In some versions, R⁷ of Formula II, Formula V, Formula VI, Formula VIII, Formula IX, or Formula X of R² is optionally substituted alkyloxy. In some versions, R⁷ of Formula II, Formula V, Formula VI, Formula VIII, Formula IX, or Formula X of R² is optionally substituted alkyl.

In some versions, R⁵ of Formula II, Formula VI, Formula VII, or Formula X of R² is hydrogen. In some versions, R⁶ of Formula II, Formula V, Formula VII, Formula VIII, or Formula IX of R² is hydrogen. In some versions, R⁷ of Formula II, Formula V, Formula VI, Formula VIII, Formula IX, or Formula X of R² is hydrogen. In some versions, R⁸ of Formula II, Formula V, Formula VI, Formula VII, or Formula IX of R² is hydrogen. In some versions, R⁹ of Formula II, Formula V, Formula VI, Formula VII, Formula VIII, or Formula X of R² is hydrogen. In some versions, R¹⁰ of Formula IV of R² is hydrogen. In some versions, R¹¹ of Formula III of R² is hydrogen. In some versions, R¹² of Formula III or Formula IV of R² is hydrogen. In some versions, R¹³ of Formula III or Formula IV of R² is hydrogen. In some versions, R¹⁴ of Formula III or Formula IV of R² is hydrogen. In some versions, R¹⁵ of Formula III or Formula IV of R² is hydrogen. In some versions, R¹⁶ of Formula III or Formula IV of R² is hydrogen. In some versions, R¹⁷ of Formula III or Formula IV of R² is hydrogen.

In some versions, R² is Formula II, Formula III, Formula IV, or Formula VI. In some versions, R² is Formula II, Formula III, or Formula IV. In some versions, R² is Formula II. In some versions, R² is Formula III. In some versions, R² is Formula IV. In some versions, R² is Formula VI.

In some versions, R² is hydrogen.

In some versions, R¹ and/or R² is Formula (II), Formula (III), Formula (IV), Formula (V), Formula (VI), Formula (VII), Formula (VIII), Formula (IX), or Formula (X) with any of the characteristics described above for R¹ and/or R². In some versions, R¹ and/or R² is Formula II, Formula III, Formula IV, or Formula VI with any of the characteristics described above for R¹ and/or R². In some versions, R¹ and/or R² is Formula II, Formula III, or Formula IV with any of the characteristics described above for R¹ and/or R². In some versions, R¹ and/or R² is Formula II with any of the characteristics described above for R¹ and/or R². In some versions, R¹ and/or R² is Formula III with any of the characteristics described above for R¹ and/or R². In some versions, R¹ and/or R² is Formula IV with any of the characteristics described above for R¹ and/or R². In some versions, R¹ and/or R² is Formula VI with any of the characteristics described above for R¹ and/or R².

In some versions, R³ is optionally substituted alkyl, optionally substituted cycloalkyl, Formula (II), Formula (III), Formula (IV), Formula (V), Formula (VI), Formula (VII), Formula (VIII), Formula (IX), or Formula (X) wherein R⁵—R¹⁷ of R³ are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

In some versions, at least one of R⁵—R⁹ of Formula II, Formula V, Formula VI, Formula VII, Formula VIII, Formula IX, or Formula X of R³ is not hydrogen. In some versions, at least one of R⁵—R⁹ of Formula II, Formula V, Formula VI, Formula VII, Formula VIII, Formula IX, or Formula X of R³ is halogen, optionally substituted alkyloxy, or optionally substituted alkyl. In some versions, at least one of R⁵—R⁹ of Formula II, Formula V, Formula VI, Formula VII, Formula VIII, Formula IX, or Formula X of R³ is halogen or optionally substituted alkyloxy. In some versions, at least one of R⁵—R⁹ of Formula II, Formula V, Formula VI, Formula VII, Formula VIII, Formula IX, or Formula X of R³ is halogen. In some versions, at least one of R⁵—R⁹ of Formula II, Formula V, Formula VI, Formula VII, Formula VIII, Formula IX, or Formula X of R³ is optionally substituted alkyloxy. In some versions, at least one of R⁵—R⁹ of Formula II, Formula V, Formula VI, Formula VII, Formula VIII, Formula IX, or Formula X of R³ is optionally substituted alkyl.

In some versions, at least one of R⁶—R⁸ of Formula II, Formula V, Formula VI, Formula VII, Formula VIII, Formula IX, or Formula X of R³ is not hydrogen. In some versions, at least one of R⁶—R⁸ of Formula II, Formula V, Formula VI, Formula VII, Formula VIII, Formula IX, or Formula X of R³ is halogen, optionally substituted alkyloxy, or optionally substituted alkyl. In some versions, at least one of R⁶—R⁸ of Formula II, Formula V, Formula VI, Formula VII, Formula VIII, Formula IX, or Formula X of R³ is halogen or optionally substituted alkyloxy. In some versions, at least one of R⁶—R⁸ of Formula II, Formula V, Formula VI, Formula VII, Formula VIII, Formula IX, or Formula X of R³ is halogen. In some versions, at least one of R⁶—R⁸ of Formula II, Formula V, Formula VI, Formula VII, Formula VIII, Formula IX, or Formula X of R³ is optionally substituted alkyloxy. In some versions, at least one of R⁶—R⁸ of Formula II, Formula V, Formula VI, Formula VII, Formula VIII, Formula IX, or Formula X of R³ is optionally substituted alkyl.

In some versions, at least one of R⁶ or R⁸ of Formula II, Formula V, Formula VI, Formula VII, Formula VIII, or Formula IX of R³ is not hydrogen. In some versions, at least one of R⁶—R⁸ of Formula II, Formula V, Formula VI, Formula VII, Formula VIII, or Formula IX of R³ is halogen, optionally substituted alkyloxy, or optionally substituted alkyl. In some versions, at least one of R⁶—R⁸ of Formula II, Formula V, Formula VI, Formula VII, Formula VIII, or Formula IX of R³ is halogen or optionally substituted alkyloxy. In some versions, at least one of R⁶—R⁸ of Formula II, Formula V, Formula VI, Formula VII, Formula VIII, or Formula IX of R³ is halogen. In some versions, at least one of R⁶—R⁸ of Formula II, Formula V, Formula VI, Formula VII, Formula VIII, or Formula IX of R³ is optionally substituted alkyloxy. In some versions, at least one of R⁶—R⁸ of Formula II, Formula V, Formula VI, Formula VII, Formula VIII, or Formula IX of R³ is optionally substituted alkyl.

In some versions, R⁷ of Formula II, Formula V, Formula VI, Formula VIII, Formula IX, or Formula X of R³ is not hydrogen. In some versions, R⁷ of Formula II, Formula V, Formula VI, Formula VIII, Formula IX, or Formula X of R³ is halogen, optionally substituted alkyloxy, or optionally substituted alkyl. In some versions, R⁷ of Formula II, Formula V, Formula VI, Formula VIII, Formula IX, or Formula X of R³ is halogen or optionally substituted alkyloxy. In some versions, R⁷ of Formula II, Formula V, Formula VI, Formula VIII, Formula IX, or Formula X of R³ is halogen. In some versions, R⁷ of Formula II, Formula V, Formula VI, Formula VIII, Formula IX, or Formula X of R³ is optionally substituted alkyloxy. In some versions, R⁷ of Formula II, Formula V, Formula VI, Formula VIII, Formula IX, or Formula X of R³ is optionally substituted alkyl.

In some versions, R⁵ of Formula II, Formula VI, Formula VII, or Formula X of R³ is hydrogen. In some versions, R⁶ of Formula II, Formula V, Formula VII, Formula VIII, or Formula IX of R³ is hydrogen. In some versions, R⁷ of Formula II, Formula V, Formula VI, Formula VIII, Formula IX, or Formula X of R³ is hydrogen. In some versions, R⁸ of Formula II, Formula V, Formula VI, Formula VII, or Formula IX of R³ is hydrogen. In some versions, R⁹ of Formula II, Formula V, Formula VI, Formula VII, Formula VIII, or Formula X of R³ is hydrogen. In some versions, R¹⁰ of Formula IV of R³ is hydrogen. In some versions, R¹¹ of Formula III of R³ is hydrogen. In some versions, R¹² of Formula III or Formula IV of R³ is hydrogen. In some versions, R¹³ of Formula III or Formula IV of R³ is hydrogen. In some versions, R¹⁴ of Formula III or Formula IV of R³ is hydrogen. In some versions, R¹⁵ of Formula III or Formula IV of R³ is hydrogen. In some versions, R¹⁶ of Formula III or Formula IV of R³ is hydrogen. In some versions, R¹⁷ of Formula III or Formula IV R³ is hydrogen.

In some versions, R³ is Formula (II), Formula (III), Formula (IV), Formula (V), Formula (VI), Formula (VII), Formula (VIII), Formula (IX), or Formula (X). In some versions, R³ is Formula II, Formula III, or Formula IV. In some versions, R³ is Formula II. In some versions, R³ is Formula III. In some versions, R³ is Formula IV.

In some versions, each R¹—R¹⁷, unless otherwise defined, is hydrogen.

In some versions, the compound is any one of the following, or a salt and/or enantiomer thereof:

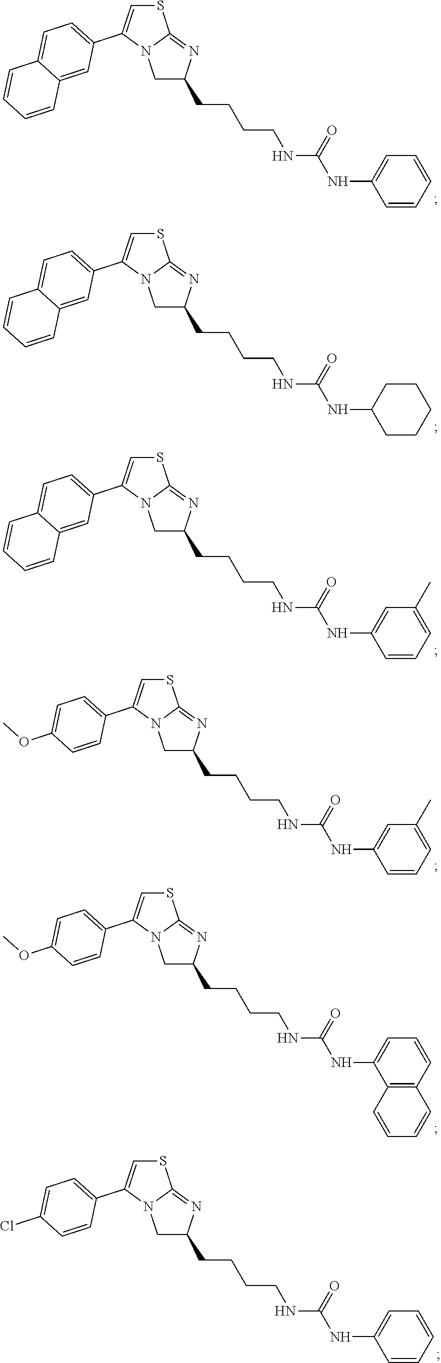

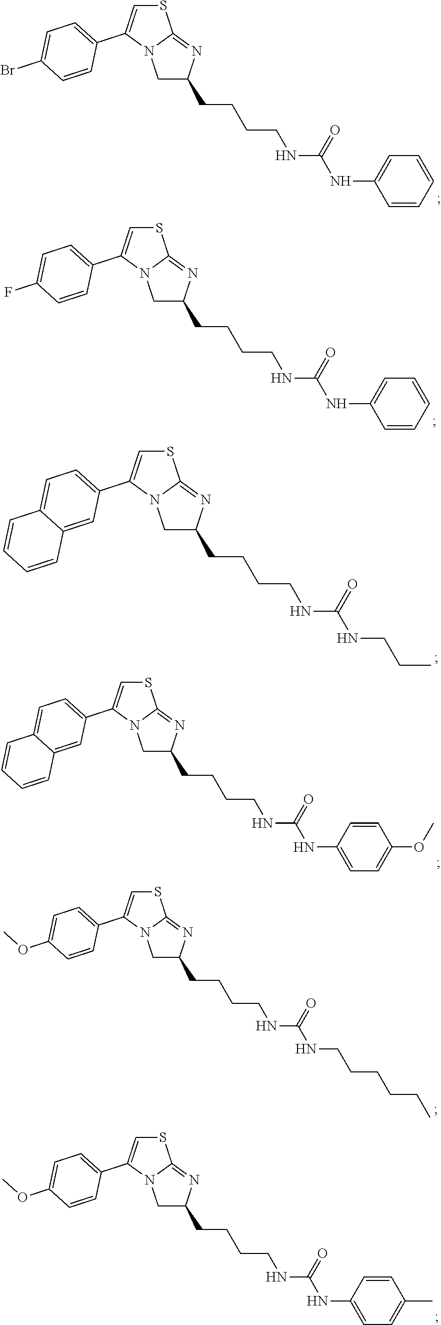

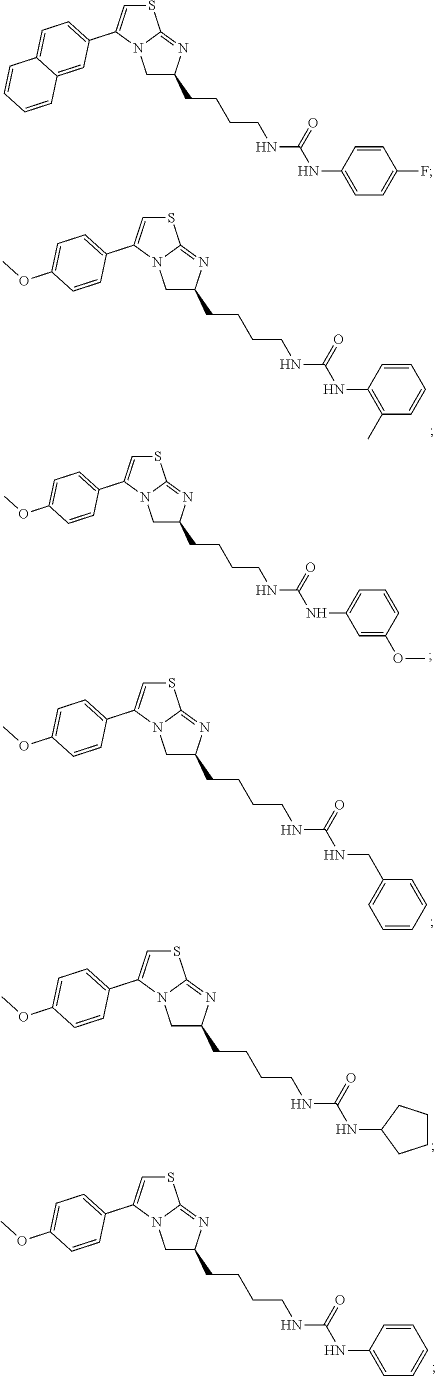

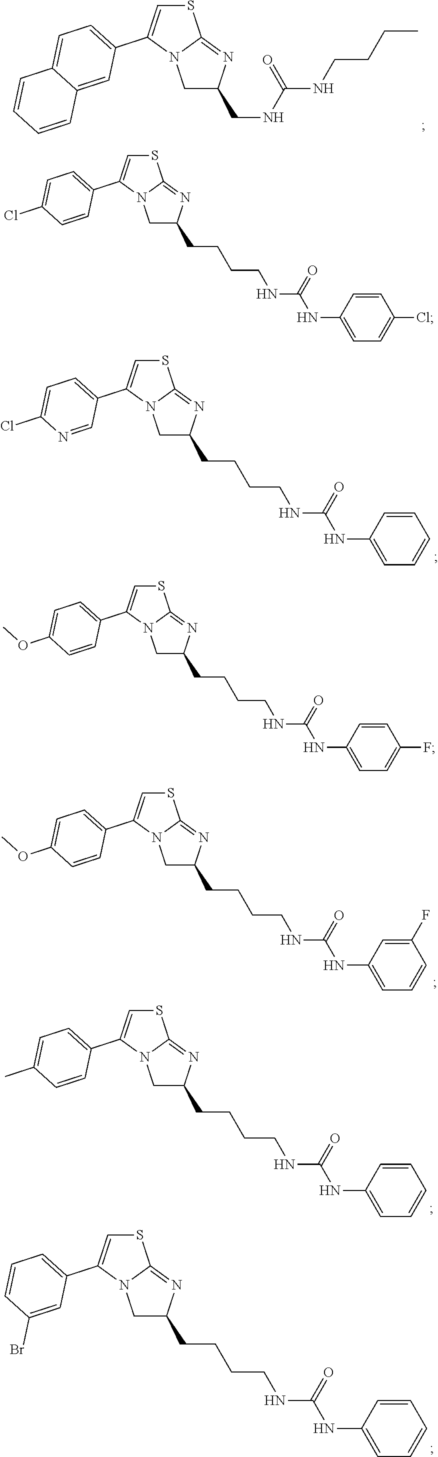

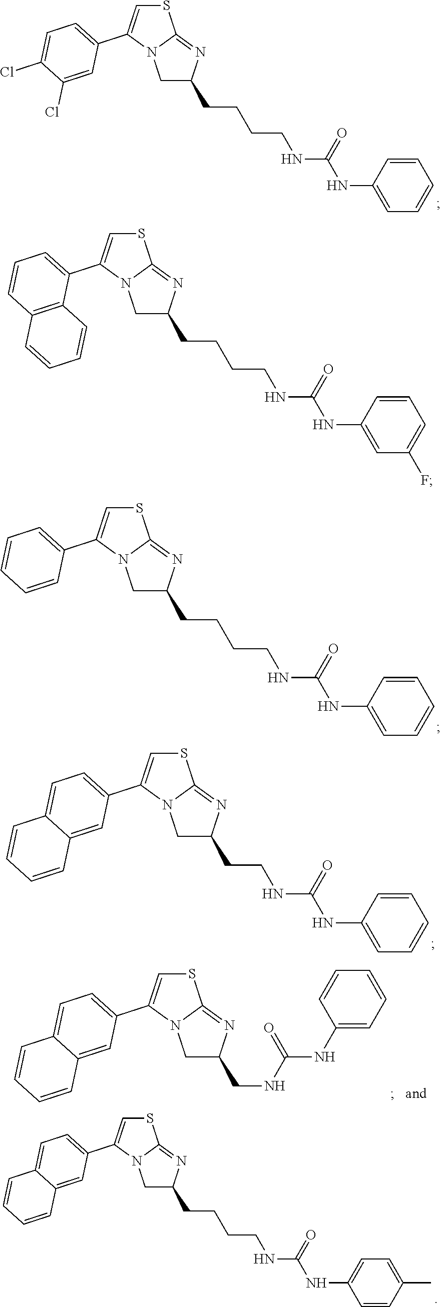

In some versions, the enantiomer is an R-enantiomer. In some versions, the enantiomer is an S-enantiomer. Compositions of the invention can comprise only the R-enantiomer, only the S-enantiomer, or a mixture of the R-enantiomer and the S-enantiomer.

Another aspect of the invention is directed to methods of inhibiting indoleamine 2,3-dioxygenase 1 (IDO1) and/or tryptophan 2,3-dioxygenase (TDO). In some versions, the methods comprise contacting the IDO1 and/or the TDO with a compound of the invention in an amount effective to inhibit the IDO1 and/or the TDO. In some versions, contacting is performed in vivo. In some versions, the contacting is performed in vitro.

Another aspect of the invention is directed to methods of treating a condition mediated by indoleamine 2,3-dioxygenase 1 (IDO1) and/or tryptophan 2,3-dioxygenase (TDO) in a subject. In some versions, the methods comprising administering a compound of the invention to the subject in an amount effective to treat the condition. In some versions, the condition is cancer. In some versions, the condition is breast cancer. In some versions, the condition is neuropathic pain.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Effects of USCYL01 on mouse body weight (A), tumor growth (B), and immune cells in tumors (C).

FIG. 2. Analgesic effect of USCYL01. Paclitaxel: Paclitaxel treatment. Paclitaxel L Y1: Paclitaxel+USCYL01 treatment.

### DETAILED DESCRIPTION OF THE INVENTION

The following definitions apply to the compounds, compositions, methods, and uses described herein unless the context clearly indicates otherwise, and it is to be understood that the claims may be amended to include language within a definition as needed or desired. Moreover, the definitions apply to linguistic and grammatical variants of the defined terms (e.g., the singular and plural forms of a term), and some linguistic variants are particularly mentioned below (e.g., “administration” and “administering”).

The term “halogen” refers to fluorine, chlorine, bromine, and iodine. Fluorine, chlorine, and bromine are preferred.

The term “hetero atom” refers to an oxygen atom, a sulfur atom, and a nitrogen atom.

The term “alkyl” includes a monovalent straight or branched hydrocarbon group having one to eight carbon atom(s). Examples include methyl, ethyl, n-propyl, isopropyl, n-butyl, isobutyl, sec-butyl, tert-butyl, n-pentyl, isopentyl, neo-pentyl, n-hexyl, isohexyl, n-heptyl, n-octyl, and the like. C1-C6 alkyl is preferred. C1-C4 alkyl or C1-C3 alkyl is further preferred. When a number of carbons is specified, it means “alkyl” having the carbon number within the range.

The term “alkenyl” includes a monovalent straight or branched hydrocarbon group having two to eight carbon atoms and one or more double bond(s). Examples include vinyl, allyl, 1-propenyl, 2-butenyl, 2-pentenyl, 2-hexenyl, 2-heptenyl, 2-octenyl, and the like. C2-C6 alkenyl is preferred. C2-C4 alkenyl is further preferred.

The term “alkynyl” includes a monovalent straight or branched hydrocarbon group having two to eight carbon atoms and one or more triple bond(s). Examples include ethynyl, 1-propynyl, 2-propynyl, 2-butynyl, 2-pentynyl, 2-hexynyl, 2-heptynyl, 2-octynyl, and the like. C2-C6 alkynyl is preferred. C2-C4 alkynyl is further preferred.

The term “cycloalkyl” includes a cycloalkyl having three to eight carbon atoms. Examples include cyclopropyl, cyclobutyl, cyclopentyl, cyclohexyl, cycloheptyl, cyclooctyl, and the like. C3-C6 cycloalkyl is preferred.

The term “cycloalkenyl” includes a cycloalkenyl having three to eight carbon atoms. Examples include cyclopropenyl, cyclobutenyl, cyclopentenyl, cyclohexenyl, cycloheptenyl, cycloocentyl, and the like. C3-C6 cycloalkenyl is preferred.

The term “alkyloxy” includes a group wherein an oxygen atom is substituted with one “alkyl” as described herein. Examples include methyloxy, ethyloxy, n-propyloxy, isopropyloxy, n-butyloxy, isobutyloxy, sec-butyloxy, tert-butyloxy, n-pentyloxy, isopentyloxy, 2-pentyloxy, 3-pentyloxy, n-hexyloxy, isohexyloxy, 2-hexyloxy, 3-hexyloxy, n-heptyloxy, n-octyloxy, and the like. C1-C6 alkyloxy is preferred. C1-C4 alkyloxy or C1-C3 alkyloxy is further preferred. When a number of carbons is specified, it means “alkyloxy” having the carbon number within the range.

The term “alkenyloxy” includes a group wherein an oxygen atom is substituted with one “alkenyl” as described herein. Examples include vinyloxy, allyloxy, 1-propenyloxy, 2-butenyloxy, 2-pentenyloxy, 2-hexenyloxy, 2-heptenyloxy, 2-octenyloxy, and the like. C2-C6 alkenyloxy is preferred. Moreover, C2-C4 alkenyloxy is further preferred. When a number of carbons is specified, it means “alkenyloxy” having the carbon number within the range.

The term “alkynyloxy” includes a group wherein an oxygen atom is substituted with one “alkynyl” as described herein. Examples include ethynyloxy, 1-propynyloxy, 2-propynyloxy, 2-butynyloxy, 2-pentynyloxy, 2-hexynyloxy, 2-heptynyloxy, 2-octynyloxy, and the like. C2-C6 alkynyloxy is preferred. C2-C4 alkynyloxy is further preferred. When a number of carbons is specified, it means “alkynyloxy” having the carbon number within the range.

The term “cycloalkyloxy” includes a group wherein an oxygen atom is substituted with one “cycloalkyl” as described herein. Examples include cyclopropyloxy, cyclobutyloxy, cyclopentyloxy, cyclohexyloxy, cycloheptyloxy, and cyclooctyloxy. C3-C6 cycloalkyloxy is preferred. When a number of carbons is specified, it means “cycloalkyloxy” having the carbon number within the range.

The term “cycloalkenyloxy” includes a group wherein an oxygen atom is substituted with one “cycloalkenyl” as described herein. Examples include cyclopropenyloxy, cyclobutenyloxy, cyclopentenyloxy, cyclohexenyloxy, cycloheptenyloxy, and cyclooctenyloxy. C3-C6 cycloalkenyloxy is preferred. When a number of carbons is specified, it means “cycloalkenyloxy” having the carbon number within the range.

The term “alkylthio” includes a group wherein a sulfur atom is substituted with one “alkyl” as described herein. Examples include methylthio, ethylthio, n-propylthio, isopropylthio, n-butylthio, isobutylthio, sec-butylthio, tert-butylthio, n-pentylthio, isopentylthio, 2-pentylthio, 3-pentylthio, n-hexylthio, isohexylthio, 2-hexylthio, 3-hexylthio, n-heptylthio, n-octylthio, and the like. C1-C6 Alkylthio is preferred. C1-C4 alkylthio is further preferred. C1-C3 alkylthio is further preferred. When a number of carbons is specified, it means “alkylthio” having the carbon number within the range.

The term “alkenylthio” includes a group wherein a sulfur atom is substituted with one “alkenyl” as described herein. Examples include vinylthio, allylthio, 1-propenylthio, 2-butenylthio, 2-pentenylthio, 2-hexenylthio, 2-heptenylthio, 2-octenylthio, and the like. C2-C6 Alkenylthio is preferred. C2-C4 alkylthio is further preferred. When a number of carbons is specified, it means “alkenylthio” having the carbon number within the range.

The term “alkynylthio” includes a group wherein a sulfur atom is substituted with one “alkynyl” as described herein. Examples include ethynylthio, 1-propynylthio, 2-propynylthio, 2-butynylthio, 2-pentynylthio, 2-hexynylthio, 2-heptynylthio, 2-octynylthio, and the like. C2-C6 alkynylthio is preferred. C2-C4 alkynylthio is further preferred. When a number of carbons is specified, it means “alkynylthio” having the carbon number within the range.

The term “alkylsulfinyl” includes a group wherein sulfinyl is substituted with one “alkyl” as described herein. Examples include methylsulfinyl, ethylsulfinyl, n-propylsulfinyl, isopropylsulfinyl, n-butylsulfinyl, isobutylsulfinyl, sec-butylsulfinyl, tert-butylsulfinyl, n-pentylsulfinyl, isopentylsulfinyl, 2-pentylsulfinyl, 3-pentylsulfinyl, n-hexylsulfinyl, isohexylsulfinyl, 2-hexylsulfinyl, 3-hexylsulfinyl, n-heptylsulfinyl, n-octylsulfinyl, and the like. C1-C6 alkylsulfinyl is preferred. C1-C4 alkylsulfinyl is further preferred.

The term “alkylsulfonyl” includes a group wherein sulfonyl is substituted with one “alkyl” as described herein. Examples include methylsulfonyl, ethylsulfonyl, n-propylsulfonyl, isopropylsulfonyl, n-butylsulfonyl, isobutylsulfonyl, sec-butylsulfonyl, tert-butylsulfonyl, n-pentylsulfonyl, isopentylsulfonyl, 2-pentylsulfonyl, 3-pentylsulfonyl, n-hexylsulfonyl, isohexylsulfonyl, 2-hexylsulfonyl, 3-hexylsulfonyl, n-heptylsulfonyl, n-octylsulfonyl, and the like. C1-C6 alkylsulfonyl is preferred. C1-C4 alkylsulfonyl is further preferred.

The term “alkylsulfonyloxy” includes a group wherein an oxygen atom is substituted with one “alkylsulfonyl” as described herein. Examples include methylsulfonyloxy, ethylsulfonyloxy, n-propylsulfonyloxy, isopropylsulfonyloxy, n-butylsulfonyloxy, isobutylsulfonyloxy, sec-butylsulfonyloxy, tert-butylsulfonyloxy, n-pentylsulfonyloxy, isopentylsulfonyloxy, 2-pentylsulfonyloxy, 3-pentylsulfonyloxy, n-hexylsulfonyloxy, isohexylsulfonyloxy, 2-hexylsulfonyloxy, 3-hexylsulfonyloxy, n-heptylsulfonyloxy, n-octylsulfonyloxy, and the like. C1-C6 alkylsulfonyl is preferred. C1-C4 alkylsulfonyl is further preferred.

The term “cycloalkylthio” includes a group wherein a sulfur atom is substituted with one “cycloalkyl” as described herein. Examples include cyclopropylthio, cyclobutylthio, cyclopentylthio, cyclohexylthio, cycloheptylthio, cyclooctylthio, and the like. C3-C6 cycloalkylthio is preferred. When a number of carbons is specified, it means “cycloalkylthio” having the carbon number within the range.

The term “cycloalkylsulfinyl” includes a group in which sulfinyl is substituted with one “cycloalkyl” as described herein. Examples include cyclopropylsulfinyl, cyclobutylsulfinyl, cyclopentylsulfinyl, cyclohexylsulfinyl, cycloheptylsulfinyl, and cyclooctylsulfinyl are exemplified. Preferably C3-C6 cycloalkylsulfinyl.

The term “cycloalkylsulfonyl” includes a group in which sulfonyl is substituted with one “cycloalkyl” as described herein. Examples include cyclopropylsulfonyl, cyclobutylsulfonyl, cyclopentylsulfonyl, cyclohexylsulfonyl, cycloheptylsulfonyl, and cyclooctylsulfonyl. C3-C6 cycloalkylsulfonyl is preferred.

The term “cycloalkylsulfonyloxy” includes a group in which an oxygen atom is substituted with one “cycloalkylsulfonyl” as described herein. Examples include cyclopropylsulfonyloxy, cyclobutylsulfonyloxy, cyclopentyl sulfonyloxy, cyclohexyl sulfonyloxy, cycloheptylsulfonyloxy, and cyclooctylsulfonyloxy. C6-C3 cycloalkylsulfonyloxy is preferred.

The term “cycloalkenylthio” includes a group in which a sulfur atom is substituted with one “cycloalkenyl” as described herein. Examples include cyclopropenylthio, cyclobutenylthio, cyclopentenylthio, cyclohexenylthio, cycloheptenylthio, and cyclooctenylthio. C3-C6 cycloalkenylthio is preferred. When a number of carbons is specified, it means “cycloalkenylthio” having the carbon number within the range.

The term “cycloalkenylsulfinyl” includes a group in which sulfinyl is substituted with one “cycloalkenyl” as described herein. Examples include cyclopropenylsulfinyl, cyclobutenylsulfinyl, cyclopentenylsulfinyl, cyclohexenylsulfinyl, cycloheptenylsulfinyl, and cyclooctenylsulfinyl. C3-C6 cycloalkenylsulfinyl is preferred.

The term “cycloalkenylsulfonyl” includes a group in which sulfonyl is substituted with one “cycloalkenyl” as described herein. Examples include cyclopropenylsulfonyl, cyclobutenylsulfonyl, cyclopentenylsulfonyl, cyclohexenylsulfonyl, cycloheptenylsulfonyl, and cyclooctenylsulfonyl. Preferably C3-C6 cycloalkenylsulfonyl is preferred.

The term “cycloalkenylsulfonyloxy” includes a group in which an oxygen atom is substituted with one “cycloalkenylsulfonyl” described as described herein. Examples include cyclobutenylsulfonyloxy, cyclopentenylsulfonyloxy, cyclopropenylsulfonyloxy, cyclohexenylsulfonyloxy, cycloheptenylsulfonyloxy, and cyclooctenylsulfonyloxy. C3-C6 cycloalkenylsulfonyloxy is preferred.

The term “alkyloxycarbonyl” includes a group in which carbonyl is substituted with one “alkyloxy” as described herein. Examples include methyloxycarbonyl, ethyloxycarbonyl, n-propyloxycarbonyl, isopropyloxycarbonyl, n-butyloxycarbonyl, tert-butyloxycarbonyl, and n-pentyloxycarbonyl. C1-C6, C1-C4, or C1-C3 alkyloxycarbonyl is preferred. C1-C2 alkyloxycarbonyl is further preferred.

The term “alkenyloxycarbonyl” includes a group in which carbonyl is substituted with one “alkenyloxy” as described herein. Examples include vinyloxycarbonyl, allyloxycarbonyl, 1-propenyloxycarbonyl, 2-butenyloxycarbonyl, and 2-pentenyloxyarbonyl. C2-C6 or C2-C4 alkyloxycarbonyl is preferred.

The term “alkynyloxycarbonyl” includes a group in which carbonyl is substituted with one “alkynyloxy” as described herein. Examples include ethynyloxycarbonyl, 1-propynyloxycarbonyl, 2-propynyloxycarbonyl, 2-butynyloxyarbonyl, and 2-pentynyloxycarbonyl. C2-C6 or C2-C4 alkynyloxycarbonyl is preferred.

The term “acyl” includes alkylcarbonyl wherein the part of alkyl is “alkyl” as described herein, alkenylcarbonyl wherein the part of alkenyl is “alkenyl” as described herein, alkynylcarbonyl wherein the part of alkynyl is “alkynyl” as described herein, cycloalkylcarbonyl wherein the part of cycloalkyl is “cycloalkyl” as described herein, arylcarbonyl wherein the part of aryl is “aryl” as described herein, heteroarylcarbonyl wherein the part of heteroaryl is “heteroaryl” as described herein, and non-aromatic heterocycliccarbonyl wherein the part of non-aromatic heterocycle is “non-aromatic heterocyclyl” as described herein. “Alkyl,” “alkenyl,” “alkynyl,” “cycloalkyl,” “aryl,” “heteroaryl,” and “non-aromatic heterocyclyl” may be substituted respectively with substituent groups exemplified in “optionally substituted alkyl,” “optionally substituted alkenyl,” “optionally substituted alkynyl,” “optionally substituted cycloalkyl,” “optionally substituted aryl,” “optionally substituted heteroaryl,” and “optionally substituted non-aromatic heterocyclyl” as described herein. Examples of the acyl group include acetyl, propionyl, butyroyl, cyclohexylcarbonyl, benzoyl, pyridinecarbonyl, and the like.

The term “optionally substituted amino” includes an amino group which may be substituted with one or two group(s) of “alkyl” as described herein, “alkenyl” as described herein, “alkynyl” as described herein, “cycloalkyl” as described herein, “cycloalkynyl” as described herein, “aryl” as described herein, “heteroaryl” as described herein, “acyl” as described herein, “alkyloxycarbonyl” as described herein, “alkenyloxycarbonyl” as described herein, “alkynyloxycarbonyl” as described herein, “alkyl sulfonyl,” “alkenylsulfonyl,” “alkynylsulfonyl,” “arylsulfonyl,” and/or “heteroarylsulfonyl” as described herein. Examples of the optionally substituted amino group include amino, methylamino, dimethylamino, ethylamino, diethylamino, ethylmethylamino, benzylamino, acetylamino, benzoylamino, methyloxycarbonylamino, and methanesulfonylamino. Amino, methylamino, dimethylamino, ethylmethylamino, diethylamino, acetylamino, and methanesulfonylamino are preferred.

The term “optionally substituted carbamoyl” includes an aminocarbonyl group wherein the part of optionally substituted amino is “optionally substituted amino” as described herein. Examples of the optionally substituted carbamoyl group includes carbamoyl, N-methylcarbamoyl, N,N-dimethylcarbamoyl, N-ethyl-N-methylcarbamoyl, N,N-diethylcarbamoyl, N-phenylcarbamoyl, N-benzylcarbamoyl, N-acetylcarbamoyl, and N-methylsulfonylcarbamoyl etc. Carbamoyl, N-methylcarbamoyl, N,N-dimethylcarbamoyl, and N-methylsulfonylcarbamoyl etc. are preferred.

The term “optionally substituted sulfamoyl” includes an aminosulfonyl group wherein the part of optionally substituted amino is “optionally substituted amino” as described herein. Examples of the optionally substituted sulfamoyl group include sulfamoyl, N-methylsulfamoyl, N,N-dimethylsulfamoyl, N-ethyl-N-methyl sulfamoyl, N,N-diethylsulfamoyl, N-phenylsulfamoyl, N-benzylsulfamoyl, N-acetylsulfamoyl, and N-methylsulfonylsulfamoyl etc. Sulfamoyl, N-methylsulfamoyl, N,N-dimethylsulfamoyl, and N-methylsulfonylsulfamoyl etc. are preferred.

The term “alkylene” means a straight or branched alkylene group. In some embodiments, the alkylene has 1-10 carbon atom(s), such as 1-6, carbon atom(s). Examples include methylene, ethylene, 1-methylethylene, trimethylene, 1-methyltrimethylene, pentamethylene, hexamethylene, and the like. C1-C4 alkylenes are preferred.

The term “aryl” includes an aromatic monocyclic or aromatic fused cyclic hydrocarbons. It may be fused with “cycloalkyl” as described herein, “cycloalkenyl” as described herein or “non-aromatic heterocyclyl” as described herein at any possible position. Both of monocyclic ring and fused ring may be substituted at any position. Examples include phenyl, 1-naphthyl, 2-naphthyl, anthryl, tetrahydronaphthyl, 1,3-benzodioxolyl, 1,4-benzodioxanyl etc. Phenyl, 1-naphthyl, and 2-naphthyl are preferred. Phenyl is further preferred.

The term “non-aromatic heterocyclyl” includes a 5- to 7-membered non-aromatic heterocyclic ring containing one or more of heteroatom(s) selected independently from oxygen, sulfur, and nitrogen atoms or a multicyclic ring formed by fusing the two or more rings thereof. Examples include pyrrolidinyl (e.g., 1-pyrrolidinyl, 2-pyrrolidinyl), pyrrolinyl (e.g., 3-pyrrolinyl), imidazolidinyl (e.g., 2-imidazolidinyl), imidazolinyl (e.g., imidazolinyl), pyrazolidinyl (e.g., 1-pyrazolidinyl, 2-pyrazolidinyl), pyrazolinyl (e.g., pyrazolinyl), piperidyl (e.g., piperidino, 2-piperidyl), piperazinyl (e.g., 1-piperazinyl), indolinyl (e.g., 1-indolinyl), isoindolinyl (e.g., isoindolinyl), morpholinyl (e.g., morpholino, 3-morpholinyl) etc.

The term “heteroaryl” includes a 5- to 6-membered aromatic ring containing one or more of heteroatom(s) selected independently from oxygen, sulfur, and nitrogen atoms. It may be fused with “cycloalkyl” as described herein, “aryl” as described herein, “non-aromatic heterocyclyl” as described herein, or other heteroaryl at any possible position. The heteroaryl group may be substituted at any position whenever it is a monocyclic ring or a fused ring. Examples include pyrrolyl (e.g., 1-pyrrolyl, 2-pyrrolyl, 3-pyrrolyl), furyl (e.g., 2-furyl, 3-furyl), thienyl (e.g., 2-thienyl, 3-thienyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), thiadiazolyl (e.g., 1,3,4-thiadiazolyl), indolidinyl (e.g., 2-indolidinyl, 6-indolidinyl), isoindolynyl (e.g., 2-isoindolynyl), indolyl (e.g., 1-indolyl, 2-indolyl, 3-indolyl), indazolyl (e.g., 3-indazolyl), purinyl (e.g., 8-purinyl), quinolidinyl (e.g., 2-quinolidinyl), isoquinolyl (e.g., 3-isoquinolyl), quinolyl (e.g., 2-quinolyl, 5-quinolyl), phtharazinyl (e.g., 1-phtharazinyl), naphthylidinyl (e.g., 2-naphthylidinyl), quinolanyl (e.g., 2-quinolanyl), quinazolinyl (e.g., 2-quinazolinyl), cinnolinyl (e.g., 3-cinnolinyl), pteridinyl (e.g., 2-pteridinyl), carbazolyl (e.g., 2-carbazolyl, 4-carbazolyl), phenanthridinyl (e.g., 2-phenanthridinyl, 3-phenanthridinyl), acridinyl (e.g., 1-acridinyl, 2-acridinyl), dibenzofuranyl (e.g., 1-dibenzofuranyl, 2-dibenzofuranyl), benzoimidazolyl (e.g., 2-benzoimidazolyl), benzoisoxazolyl (e.g., 3-benzoisoxazolyl), benzooxazolyl (e.g., 2-benzooxazolyl), benzooxadiazolyl (e.g., 4-benzooxadiazolyl), benzoisothiazolyl (e.g., 3-benzoisothiazolyl), benzothiazolyl (e.g., 2-benzothiazolyl), benzofuryl (e.g., 3-benzofuryl), benzothienyl (e.g., 2-benzothienyl), dibenzothienyl (e.g., 2-dibenzothienyl), and benzodioxolyl (e.g., 1,3-benzodioxolyl), etc.

The term “aryloxy” includes a group in which an oxygen atom is substituted with one “aryl” as described herein. Examples include phenyloxy and naphthyloxy, etc.

The term “arylthio” includes a group in which a sulfur atom is substituted with one “aryl” as described herein. Examples include phenylthio and naphthylthio, etc.

The term “arylsulfinyl” includes a group in which sulfinyl is substituted with one “aryl” as described herein. Examples include phenylsulfinyl and naphthylsulfinyl, etc.

The term “arylsulfonyl” includes a group in which sulfonyl is substituted with one “aryl” as described herein. Examples include phenylsulfonyl and naphthylsulfoinyl, etc.

Examples of “arylsulfonyloxy” include phenylsulfonyloxy and naphthylsulfonyloxy, etc.

The term “aryloxycarbonyl” includes a group in which carbonyl is substituted with one “aryloxy” as described herein. Examples include phenyloxycarbonyl, 1-naphthyloxycarbonyl and 2-naphthyloxycarbonyl, etc.

The term “heteroaryloxy” includes a group in which an oxygen atom is substituted with one “heteroaryl” as described herein. Examples include pyrrolyloxy, furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, pyridazinyloxy, tetrazolyloxy, oxadiazolyloxy, thiadiazolyloxy, indolidinyloxy, isoindolynyloxy, indolyloxy, indazolyloxy, purinyloxy, quinolidinyloxy, isoquinolyloxy, quinolyloxy, phtharazinyloxy, naphthylidinyloxy, quinolanyloxy, quinazolinyloxy, cinnolinyloxy, pteridinyloxy, carbazolyloxy, phenanthridinyloxy, acridinyloxy, dibenzofuranyloxy, benzoimidazolyloxy, benzoisoxazolyloxy, benzooxazolyloxy, benzooxadiazolyloxy, benzoisothiazolyloxy, benzothiazolyloxy, benzofuryloxy, benzothienyloxy, dibenzothicnyloxy, and benzodioxolyloxy are exemplified. Preferably furyloxy, thienyloxy, imidazolyloxy, pyrazolyloxy, isothiazolyloxy, isoxazolyloxy, oxazolyloxy, thiazolyloxy, pyridyloxy, pyrazinyloxy, pyrimidinyloxy, and pyridazinyloxy, etc.

The term “heteroarylthio” includes a group in which a sulfur atom is substituted with one “heteroaryl” as described herein. Examples include pyrrolylthio, furylthio, thienylthio, imidazolylthio, pyrazolylthio, isothiazolylthio, isoxazolylthio, oxazolylthio, thiazolylthio, pyridylthio, pyrazinylthio, pyrimidinylthio, pyridazinylthio, tetrazolylthio, oxadiazolylthio, thiadiazolylthio, indolidinylthio, isoindolynylthio, indolylthio, indazolylthio, purinylthio, quinolidinylthio, isoquinolylthio, quinolylthio, phtharazinylthio, naphthylidinylthio, quinolanylthio, quinazolinylthio, cinnolinylthio, ptcridinylthio, carbazolylthio, phenanthridinylthio, acridinylthio, dibenzofuranylthio, benzoimidazolylthio, benzoisoxazolylthio, benzooxazolylthio, benzooxadiazolylthio, benzoisothiazolylthio, benzothiazolylthio, benzofurylthio, benzothienylthio, dibenzothienylthio, and benzodioxolylthio etc. are exemplified. Preferably furylthio, thienylthio, imidazolylthio, pyrazolylthio, isothiazolylthio, isoxazolylthio, oxazolylthio, thiazolylthio, pyridylthio, pyrazinylthio, pyrimidinylthio, and pyridazinylthio, etc.

The term “heteroarylsulfinyl” includes a group in which sulfinyl is substituted with one “heteroaryl” as described herein. Examples include pyrrolylsulfinyl, furylsulfinyl, thienylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, pyridazinylsulfinyl, tetrazolylsulfinyl, oxadiazolylsulfinyl, thiadiazolylsulfinyl, indolidinylsulfinyl, isoindolylsulfinyl, indolylsulfinyl, indazolylsulfinyl, purinylsulfinyl, quinolidinylsulfinyl, isoquinolylsulfinyl, quinolylsulfinyl, phtharazinylsulfinyl, naphthylidinylsulfinyl, quinolanylsulfinyl, quinazolinylsulfinyl, cinnolinylsulfinyl, ptcridinylsulfinyl, carbazolylsulfinyl, phenanthridinylsulfinyl, acridinylsulfinyl, dibenzofuranylsulfinyl, benzoimidazolylsulfinyl, benzoisoxazolylsulfinyl, benzooxazolylsulfinyl, benzooxadiazolylsulfinyl, benzoisothiazolylsulfinyl, benzothiazolylsulfinyl, benzofurylsulfinyl, benzothicnylsulfinyl, dibenzothienylsulfinyl, and benzodioxolylsulfinyl etc. are exemplified. Preferably furylsulfinyl, thicnylsulfinyl, imidazolylsulfinyl, pyrazolylsulfinyl, isothiazolylsulfinyl, isoxazolylsulfinyl, oxazolylsulfinyl, thiazolylsulfinyl, pyridylsulfinyl, pyrazinylsulfinyl, pyrimidinylsulfinyl, and pyridazinylsulfinyl, etc.

The term “heteroarylsulfonyl” includes a group in which sulfonyl is substituted with one “heteroaryl” as described herein. Examples include pyrrolylsulfonyl, furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, pyridazinylsulfonyl, tctrazolylsulfonyl, oxadiazolylsulfonyl, thiadiazolylsulfonyl, indolizinylsulfonyl, isoindolylsulfonyl, indolylsulfonyl, indazolylsulfonyl, purinylsulfonyl, quinolidinylsulfonyl, isoquinolylsulfonyl, quinolylsulfonyl, phtharazinylsulfonyl, naphthilidinylsulfonyl, quinolanylsulfonyl, quinazolinylsulfonyl, cinnolinyl sulfonyl, pteridinyl sulfonyl, carbazolylsulfonyl, phenanthridinylsulfonyl, acridinylsulfonyl, dibenzofuranylsulfonyl, benzoimidazolylsulfonyl, benzoisoxazolylsulfonyl, benzooxazolylsulfonyl, benzooxadiazolylsulfonyl, benzoisothiazolylsulfonyl, benzothiazolylsulfonyl, benzofurylsulfonyl, benzothienylsulfonyl, dibenzothienylsulfonyl, and benzodioxolylsulfonyl, ctc. Furylsulfonyl, thienylsulfonyl, imidazolylsulfonyl, pyrazolylsulfonyl, isothiazolylsulfonyl, isoxazolylsulfonyl, oxazolylsulfonyl, thiazolylsulfonyl, pyridylsulfonyl, pyrazinylsulfonyl, pyrimidinylsulfonyl, and pyridazinylsulfonyl are preferred.

The term “heteroarylsulfonyloxy” includes a group in which an oxygen atom is substituted with one “heteroarylsulfonyl” as described herein. Examples include pyrrolylsulfonyloxy, furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, pyridazinylsulfonyloxy, tetrazolylsulfonyloxy, oxadiazolylsulfonyloxy, thiadiazolylsulfonyloxy, indolizinylsulfonyloxy, isoindolylsulfonyloxy, indolylsulfonyloxy, indazolylsulfonyloxy, purinylsulfonyloxy, quinolidinylsulfonyloxy, isoquinolylsulfonyloxy, quinolylsulfonyloxy, phtharazinylsulfonyloxy, naphthilidinylsulfonyloxy, quinolanyl sulfonyloxy, quinazolinylsulfonyloxy, cinnolinylsulfonyloxy, pteridinylsulfonyloxy, carbazolylsulfonyloxy, acridinylsulfonyloxy, dibenzofuranylsulfonyloxy, phenanthridinylsulfonyloxy, benzoisoxazolylsulfonyloxy, benzooxazolylsulfonyloxy, benzoimidazolylsulfonyloxy, benzoisothiazolylsulfonyloxy, benzothiazolylsulfonyloxy, benzooxadiazolylsulfonyloxy, benzothienylsulfonyloxy, dibenzothienylsulfonyloxy, and benzofuryl sulfonyloxy, benzodioxolylsulfonyloxy etc. are exemplified. Preferably, furylsulfonyloxy, thienylsulfonyloxy, imidazolylsulfonyloxy, pyrazolylsulfonyloxy, isothiazolylsulfonyloxy, isoxazolylsulfonyloxy, oxazolylsulfonyloxy, thiazolylsulfonyloxy, pyridylsulfonyloxy, pyrazinylsulfonyloxy, pyrimidinylsulfonyloxy, and pyridazinylsulfonyloxy, etc.

The term “aromatic carbocyclic ring” includes an aromatic monocyclic or aromatic fused carbocyclic ring. Examples include a benzene ring, a naphthalene ring, and an anthracene ring. A benzene ring is preferred.

The term “aromatic heterocyclic ring” includes an aromatic monocyclic or aromatic fused heterocyclic ring. Examples include a pyrrole ring, a furan ring, a thiophen ring, a pyrazole ring, an imidazole ring, an isothiazole ring, an isoxazole ring, an oxazole ring, a thiazole ring, a pyrazine ring, a pyrimidine ring, a pyridazine ring, a tetrazole ring, an oxadiazole ring, a thiadiazole ring, an indolizine ring, an isoindole ring, an indole ring, an indazole ring, a purine ring, a quinolidine ring, an isoquinoline ring, a quinoline ring, a phtharazine ring, a naphthyridine ring, a quinolane ring, a quinazoline ring, a cinnoline ring, a pteridine ring, a carbazole ring, a phenanthridine ring, an acridine ring, a dibenzofuran ring, a benzimidazole ring, a benzisoxazole ring, a benzoxazole ring, a benzoxadiazole ring, a benzisothiazole ring, a benzothiazole ring, a benzofuran ring, a benzothiophene ring, a dibenzothiophene ring, and a benzodixolane ring are exemplified. Preferably a pyridine ring, a furan ring, and a thiophen ring are exemplified.

The term “C1-C6 alkylene” includes a straight or branched alkylene group having one to six carbon atom(s). Examples include —CH₂—, —CH(CH₃)—, —C(CH₃)₂—, —CH₂CH₂—, CH(CH₃)CH₂—, —C(CH₃)₂CH₂—, —CH₂CH₂CH₂—, —CH₂CH₂CH₂CH₂—, —CH₂CH₂CH₂CH₂CH₂—, and —CH₂CH₂CH₂CH₂CH₂CH₂—. Preferred are —CH₂—, CH₂CH₂—, —CH₂CH₂CH₂—, and —CH₂CH₂CH₂CH₂—.

The term “alkylene optionally containing one or more heteroatom(s)” of “optionally substituted alkylene optionally containing one or more heteroatom(s)” includes a straight or branched alkylene group, optionally containing one or more heteroatom(s). The straight or branched alkylene group in some embodiments can have 1-10, 1-6, 10, 9, 8, 7, 6, 5, 4, 3, 2, or 1 carbon atoms. The one or more heteroatom(s) in some embodiments is one or two heteroatom(s). The heteroatom(s) may be included internally within the alkylene group or constitute one or both terminal ends of the alkylene group. The heteroatom(s) are preferably included only internally within the alkylene group and do not constitute either of the terminal ends. Any included heteroatom(s) do not count toward the number of specified carbon atoms. The optional substitutions can be R⁴ as described herein, such as “alkyl” as described herein. Examples of “alkylene optionally containing one or more heteroatom(s)” include —CH₂—, —CH(CH₃)—, —C(CH₃)₂—, CH₂CH₂—, CH₂CH₂CH₂—-, —CH₂CH₂CH₂CH₂—, —CH₂CH₂CH₂CH₂CH₂—, —CH₂CH₂CH₂CH₂CH₂CH₂—, —CH₂O—, —OCH₂—, —CH₂CH₂O—, —OCH₂CH₂, —CH₂S—, —SCH₂—, —CH₂CH₂S—, —SCH₂CH₂—, —CH₂CH₂OCH₂CH₂—, —OCH₂CH₂O—, —OCH₂O—, —NHCH₂—, —N(CH₃)CH₂—, —N⁺(CH₃)₂CH₂—, —NHCH₂CH₂CH₂—, and —N(CH₃)CH₂CH₂CH₂—, etc. Preferred are —CH₂—, —CH₂CH₂—, —CH₂CH₂CH₂—, —CH₂CH₂CH₂CH₂—, —OCH₂CH₂O—, —OCH₂O—, and —N(CH₃)CH₂CH₂CH₂—.

The term “alkenylene optionally containing one or two heteroatom(s)” of “optionally substituted alkenylene optionally containing one or two heteroatom(s)” includes a straight or branched alkenylene group having two to six carbon atoms, optionally containing one or two heteroatom(s) which may be substituted with “alkyl” as described herein. Examples include CH═CHCH═CH—, —CH—CHO—, —OCH—CH—, —CH═CHS—, —SCH═CH—, CH═CHNH—, —NHCH—CH—, —CH—CH—CH—N—, and —N═CH—CH═CH—. Preferred are, —CH═CHCH═CH—, —CH—CHCH—N—, and —N═CHCH═CH—.

The term “alkynylene optionally containing one or two heteroatom(s)” includes a straight or branched alkynylene group having two to six carbon atoms, optionally containing one or two heteroatom(s) which may be substituted with “alkyl” as described herein. Examples include C≡CCH₂—, —CH₂C≡CCH₂—, —CH₂C≡CCH₂O—, —OCH₂C≡CH—, —CH₂C≡CCH₂S—, —SCH₂C≡CH—, —CH₂C≡CCH₂NH—, —NHCH₂C≡CH—, —CH₂C≡CCH₂N (CH₃)—, and —N(CH₃)CH₂C≡CH—. Especially, —CH₂C≡CCH₂—, and —OCH₂C≡CH—are preferred.

The term “3- to 8-membered nitrogen-containing non-aromatic heterocyclic ring” includes a ring of any of the formulas described as such in U.S. Pat. No. 8,143,285, which is incorporated herein by reference in its entirety.

The term “3- to 8-nitrogen-containing aromatic heterocyclic ring” includes a 3- to 8-membered aromatic heterocyclic ring containing one or more of nitrogen atom(s), and further optionally an oxygen atom and/or sulfur atom in the ring. Examples include pyrrolyl (e.g., 1-pyrrolyl, 2-pyrrolyl, 3-pyrrolyl), imidazolyl (e.g., 2-imidazolyl, 4-imidazolyl), pyrazolyl (e.g., 1-pyrazolyl, 3-pyrazolyl), isothiazolyl (e.g., 3-isothiazolyl), isoxazolyl (e.g., 3-isoxazolyl), oxazolyl (e.g., 2-oxazolyl), thiazolyl (e.g., 2-thiazolyl), pyridyl (e.g., 2-pyridyl, 3-pyridyl, 4-pyridyl), pyrazinyl (e.g., 2-pyrazinyl), pyrimidinyl (e.g., 2-pyrimidinyl, 4-pyrimidinyl), pyridazinyl (e.g., 3-pyridazinyl), tetrazolyl (e.g., 1H-tetrazolyl), oxadiazolyl (e.g., 1,3,4-oxadiazolyl), and thiadiazolyl (e.g., 1,3,4-thiadiazolyl).

The term “4- to 8-membered nitrogen-containing heterocyclic ring containing one or two nitrogen atom(s)” means a ring of any of the formulas described as such in U.S. Pat. No. 8,143,285, which is incorporated herein by reference in its entirety.

The term “oxo” refers to an—O group.

“Optionally substituted” is used interchangeably herein with “substituted or unsubstituted.”

In the present specification, examples of substituents in “optionally substituted alkyl,” “optionally substituted alkyloxy,” “optionally substituted alkylthio,” “optionally substituted alkylsulfinyl,” “optionally substituted alkylsulfonyl,” “optionally substituted alkylsulfonyloxy,” and “optionally substituted alkyloxycarbonyl” include cycloalkyl, alkylene optionally containing one or two heteroatom(s), hydroxyl, oxo, alkyloxy optionally substituted with a substituent group A at one to three position(s), thiol, alkylthio, halogen, nitro, cyano, carboxyl, sulfino (—SO₂H), alkyloxycarbonyl, optionally substituted amino, optionally substituted carbamoyl, acyl, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), an optionally substituted non-aromatic heterocyclic ring group (e.g., morpholinyl, pyrrolidinyl, piperazinyl) which may be substituted with a substituent group C at one to three position(s), aryloxy (e.g., phenyloxy) optionally substituted with a substituent group B at one to three position(s), alkylsulfonyl, and the like. The above-referenced “optionally substituted” moieties can be substituted with one to three of the above-referenced substituent(s) at any possible position.

In the present specification, examples of substituents in “optionally substituted alkenyl,” “optionally substituted alkynyl,” “optionally substituted alkenyloxy,” “optionally substituted alkynyloxy,” “optionally substituted alkenylthio,” “optionally substituted alkynylthio,” “optionally substituted alkenyloxycarbonyl,” “optionally substituted alkynyloxycarbonyl,” “optionally substituted cycloalkyl,” “optionally substituted cycloalkenyl,” “optionally substituted cycloalkyloxy, “optionally substituted cycloalkenyloxy,” “optionally substituted cycloalkylthio,” “optionally substituted cycloalkenylthio,” “optionally substituted cycloalkylsulfinyl,” “optionally substituted cycloalkenylsulfinyl,” “optionally substituted cycloalkylsulfonyl,” “optionally substituted cycloalkenylsulfonyl,” “optionally substituted cycloalkylsulfonyloxy,” “optionally substituted cycloalkenylsulfonyloxy,” “optionally substituted alkenyloxycarbonyl,” “optionally substituted alkylene,” “optionally substituted C1-C6 alkylene,” “optionally substituted alkylene optionally containing one or two heteroatom(s),” “optionally substituted alkenylene,” “optionally substituted alkenylene optionally containing one or two heteroatom(s),” “optionally substituted alkynylene,” and “optionally substituted alkynylene optionally containing one or two heteroatom(s)” include alkyl (such as dialkyl) optionally substituted with a substituent group D at one to three position(s), cycloalkyl, hydroxyl, oxo, alkyloxy optionally substituted with a substituent group A at one to three position(s), thiol, alkylthio, halogen, nitro, cyano, carboxyl, sulfino, alkyloxycarbonyl, optionally substituted amino, optionally substituted carbamoyl, acyl, acyloxy, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), non-aromatic heterocyclyl (e.g., morpholinyl, pyrrolidinyl, piperazinyl) optionally substituted with a substituent group C at one to three position(s), aryloxy (e.g., phenyloxy) optionally substituted with a substituent group C at one to three position(s), alkylsulfonyl, and the like. The above-referenced “optionally substituted” moieties can be substituted with one or more of the above-referenced substituent(s) at any possible position.

In the present specification, examples of substituents in “optionally substituted aryl,” “optionally substituted phenoxy,” “optionally substituted aryloxy,” “optionally substituted phenylthio,” “optionally substituted arylthio,” “optionally substituted arylsulfinyl,” “optionally substituted arylsulfonyl,” “optionally substituted arylsulfonyloxy,” “optionally substituted heteroaryl,” “optionally substituted heteroaryloxy,” “optionally substituted heteroarylthio,” “optionally substituted heteroarylsulfinyl,” “optionally substituted heteroarylsulfonyl,” “optionally substituted heteroarylsulfonyloxy,” “optionally substituted non-aromatic heterocyclyl,” “optionally substituted C6 arene-1,4-diamine-N1,N⁴-diyl,” and substituted C6 arene-1,4-diamine-N1,N⁴-diyl,” include alkyl optionally substituted with a substituent group D at one to three position(s), cycloalkyl, alkenyl, alkynyl, hydroxyl, alkyloxy optionally substituted with a substituent group A at one to three position(s), aryloxy (e.g., phenoxy) optionally substituted with a substituent group B at one to three position(s), thiol, alkylthio, halogen, nitro, cyano, carboxyl, sulfino, alkyloxycarbonyl, acyl, alkylsulfonyl, optionally substituted amino, optionally substituted carbamoyl, aryl (e.g., phenyl) optionally substituted with a substituent group B at one to three position(s), heteroaryl (e.g., pyridyl, furyl, thienyl, imidazolyl, oxazolyl, thiazolyl, pyrazolyl) optionally substituted with a substituent group C at one to three position(s), non-aromatic heterocyclyl (e.g., morpholinyl, pyrrolidinyl, piperazinyl) optionally substituted with a substituent group C at one to three position(s), and the like. The above-referenced “optionally substituted” moieties can be substituted with one or more of the above-referenced substituent(s) at any possible position.

Substituent group A is comprised of halogen and phenyl optionally substituted with one to three substituent(s) selected from the Substituent group B.

Substituent group B is comprised of halogen, alkyl, alkyloxy, cyano, and nitro.

Substituent group C is comprised of halogen and alkyl.

Substituent group D is comprised of halogen and alkyloxy.

“- - -” between adjacent atoms indicates a bond that is present or absent depending on the valency of the adjacent atoms in a given specified structural context. The bond may comprise localized electrons between the adjacent atoms or delocalized electrons depending on the given specified structural context.

“Not hydrogen” used to define any variable (e.g., R¹—R¹⁷) means that the variable can be any moiety defined for the variable except for hydrogen. For example, in some cases, the term “not hydrogen” defines the variable as halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

The terms “compound(s) of the invention” and “inhibitor(s) of the invention” are used interchangeably and refer to compounds of Formula (I) as described herein.

The terms “inhibit” and “inhibiting” used with reference to inhibiting indolcamine 2,3-dioxygenase 1 (IDO1) and/or tryptophan 2,3-dioxygenase (TDO) refers to reducing the activity of IDO1 and/or TDO to any extent.

Contacting IDO1 and/or TDO with a compound of the invention can be performed in vitro or in vivo. When performed in vivo, the compound can be administered to a subject such that the compound is delivered to the IDO1 and/or TDO in vivo.

A “condition mediated” by a certain factor (e.g., an enzyme such as IDO1 and/or TDO) or pathway (e.g., the kynurenine pathway) refers to any condition in which the factor or pathway contributes to or is involved in the etiology or maintenance of the condition. IDO1 and/or TDO are well known in the art to mediate a large number of conditions. These conditions include kynurenine pathway-mediated conditions, as IDO1 and TDO catalyze the initial rate-limiting step of the kynurenine pathway. Examples of IDO1- and/or TDO-mediated conditions include cancer, such as breast cancer, and neuropathic pain (see following Examples). Other examples of IDO1-and/or TDO-mediated conditions include inflammation and inflammatory diseases such as infection, coronary heart disease, autoimmune syndrome, cancer, and neurodegenerative disorders (Strasser, B.; Becker, K.; Fuchs, D.; Gostner, J. M. Kynurenine pathway metabolism and immune activation: Peripheral measurements in psychiatric and co-morbid conditions. Neuropharmacology 2017, 112, 286-296); coronary heart disease (Chen, Y.; Guillemin, G. J. Kynurenine pathway metabolites in humans: Disease and healthy States. Int. J. Tryptophan Res. 2009, 2, 1-19), depression and/or schizophrenia (Cervenka, I.; Agudelo, L. Z.; Ruas, J. L. Kynurenines: Tryptophan's metabolites in exercise, inflammation, and mental health. Science 2017, 357, caaf9794); immune-related diseases, such as inflammatory bowel disease, cardiovascular disease, osteoporosis, and/or polycystic ovary syndrome (Tsuji A, Ikeda Y, Yoshikawa S, Taniguchi K, Sawamura H, Morikawa S, Nakashima M, Asai T, Matsuda S. The Tryptophan and Kynurenine Pathway Involved in the Development of Immune-Related Diseases. Int J Mol Sci. 2023 March 17; 24 (6): 5742); and Alzheimer's disease, amyotrophic lateral sclerosis, Huntington's disease, HIV, AIDS, dementia, malaria, cancer, depression, schizophrenia, rheumatoid arthritis, epilepsy, multiple sclerosis, cerebrovascular disease, epilepsy, anorexia nervosa, hematological neoplasias, anxiety, streptococcal toxic shock syndrome, colorectal cancer, coronary heart disease, gynecological cancer, osteoporosis, chronic brain injury, and stroke (Chen Y, Guillemin G J. Kynurenine pathway metabolites in humans: disease and healthy States. Int J Tryptophan Res. 2009; 2:1-19; among others.

The term “about,” when used in reference to a value, signifies any value or range of values that is plus- or-minus 10% of the stated value (e.g., within plus- or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). As another example, a prevalence rank in a population of about 80% means a prevalence rank of 72-88% (e.g., 79.2-80.8%). In case of doubt, “about X” can be “X” (e.g., about 80% can be 80%). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure, etc.)). In the event an instrument or technique measuring a value has a margin of error greater than 10%, a given value will be about the same as a stated value when they are both within the margin of error for that instrument or technique.

The term “administration” and variants thereof, such as “administering,” refer to the administration of a compound described herein, or a composition containing the compound to a subject (e.g., a human patient) or system (e.g., a cell- or tissue-based system that is maintained ex vivo). As a result of the administration, the compound or composition containing the compound (e.g., a pharmaceutical composition) is introduced to the subject or system. One of ordinary skill in the art will be aware of a variety of routes that can, in appropriate circumstances, be utilized for administration to a subject or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours).

The term “cancer” refers to a disease in which biological cells exhibit an aberrant growth phenotype characterized by loss of control of cell proliferation to an extent that will be detrimental to a patient having the disease. A cancer can be classified by the type of tissue in which it originated (histological type) and/or by the primary site in the body in which the cancer first developed. Based on histological type, cancers are generally grouped into six major categories: carcinomas; sarcomas; myelomas; leukemias; lymphomas; and mixed types. A cancer treated as described herein may be of any one of these types and may comprise cells that are precancerous (e.g., benign), malignant, pre-metastatic, metastatic, and/or non-metastatic. A patient who has a malignancy or malignant lesion has a cancer. The present disclosure specifically identifies certain cancers to which its teachings may be particularly relevant.

One of ordinary skill in the art will appreciate that the term “dosage form” may be used to refer to a physically discrete unit of a compound of the for administration to a patient. Typically, each such unit contains a predetermined quantity of the compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

One of ordinary skill in the art will appreciate that the term “dosing regimen” may be used to refer to a set of unit doses (typically more than one) that are administered individually to a patient, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of a compound of the invention refers to an amount that produces or is expected to produce the desired effect for which it is administered. The effective amount will vary depending on factors such as the desired biological endpoint, the pharmacokinetics of the compound administered, the condition being treated, the mode of administration, and characteristics of the patient, as discussed further below and recognized in the art. The term can be applied to therapeutic and prophylactic methods. For example, a therapeutically effective amount is one that reduces the incidence and/or severity of one or more signs or symptoms of the disease. For example, in treating a cancer, an effective amount may reduce the tumor burden, stop tumor growth, inhibit metastasis, or prolong patient survival. One of ordinary skill in the art will appreciate that the term does not in fact require successful treatment be achieved in any particular individual. Rather, a therapeutically effective amount is that amount that provides a particular desired pharmacological response in a significant number of patients when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses, for example, as part of a dosing regimen.

The terms “patient” and “subject” are used interchangeably herein and refer to any organism that is or may be subjected to the administration of a compound of the invention. The administration may be administered for, e.g., experimental, diagnostic, prophylactic, and/or therapeutic purposes. Typical patients include animals (e.g., mammals such as mice, rats, rabbits, non-human primates, and humans; domesticated animals, such as dogs and cats; and livestock or any other animal of agricultural or commercial value). A patient may be suffering from or be susceptible to (i.e., have a higher than average risk of developing) a disease described herein and may display one or more signs or symptoms thereof.

The term “pharmaceutically acceptable,” when applied to a carrier used to formulate a composition disclosed herein (e.g., a pharmaceutical composition), means a carrier that is compatible with the other ingredients of the composition and not deleterious to a patient (e.g., it is non-toxic in the amount required and/or administered (e.g., in a unit dosage form)).

The term “pharmaceutically acceptable,” when applied to a salt, solvate, stereoisomer, tautomer, or isotopic form of a compound described herein, refers to a salt, solvate, stereoisomer, tautomer, or isotopic form that is, within the scope of sound medical judgment, suitable for use in contact with the tissues of humans (e.g., patients) and lower animals (including, but not limited to, mice and rats used in laboratory studies) without unacceptable toxicity, irritation, allergic response and the like, and that can be used in a manner commensurate with a reasonable benefit/risk ratio. Many pharmaceutically acceptable salts are well known in the art. Pharmaceutically acceptable salts of the compounds described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C14 alkyl) 4 salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “reference” used with regard to an agent, patient, population, sample, sequence, or value of interest refers to a standard or control relative to which a comparison is performed. For example, an agent, patient, population, sample, sequence, or value of interest is compared with a reference agent, patient, population, sample, sequence, or value. The reference can be analyzed or determined substantially simultaneously with the analysis or determination of the item of interest, or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One of ordinary skill in the art will appreciate when sufficient similarities are present to justify reliance on and/or comparison to a particular possible reference as a standard or control.

As used herein, a “response” to treatment is any beneficial alteration in a patient's condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, etc. A response may be a patient's response or a tumor's response.

As used herein, the terms “treatment,” “treat,” and “treating” refer to reversing, alleviating, delaying the onset of, and/or inhibiting the progress of a “pathological condition” (e.g., a disease, such as cancer) described herein. In some embodiments, “treatment,” “treat,” and “treating” require that signs or symptoms of the disease have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

Relative amounts of the compound(s) of the invention, the pharmaceutically acceptable carrier(s), and/or any additional ingredients in a pharmaceutical composition of the invention can vary, depending upon the identity, size, and/or condition of the subject treated and further depending upon the route by which the composition is to be administered and the disease to be treated. By way of example, the composition may comprise between about 0.1% and 99.9% (w/w or w/v) of a compound of the invention.

Pharmaceutically acceptable carriers useful in the manufacture of the pharmaceutical compositions described herein are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable carriers useful in the manufacture of the pharmaceutical compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Pharmaceutical compositions used as described herein may be administered orally. Such orally acceptable dosage forms may be solid (e.g., a capsule, tablet, sachet, powder, granule, and orally dispersible film) or liquid (e.g., an ampoule, semi-solid, syrup, suspension, or solution (e.g., aqueous suspensions or dispersions and solutions). In the case of tablets, carriers commonly used include lactose and corn starch. Lubricating agents, such as magnesium stearate, can also be included. In the case of capsules, useful diluents include lactose and dried cornstarch. When aqueous suspensions are formulated, the compound can be combined with emulsifying and suspending agents. In any oral formulation, sweetening, flavoring, or coloring agents may also be added. In any of the various embodiments described herein, an oral formulation can be formulated for immediate release or sustained/delayed release and may be coated or uncoated. A provided composition can also be micro-encapsulated.

Compositions suitable for buccal or sublingual administration include tablets, lozenges, and pastilles. Formulations can also be prepared for subcutaneous, intravenous, intramuscular, intraocular, intravitreal, intra-articular, intra-synovial, intrasternal, intrathecal, intrahepatic, intraperitoneal intralesional and by intracranial injection or infusion techniques. Preferably, the compositions are administered orally, subcutaneously, intraperitoneally or intravenously. Sterile injectable forms of the compositions of this invention may be aqueous or oleaginous suspension.

These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Although the descriptions of pharmaceutical compositions provided herein are principally directed to pharmaceutical compositions which are suitable for administration to humans, it will be understood by one of ordinary skill in the art that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification.

Compounds described herein are typically formulated in dosage unit form, e.g., single unit dosage form, for case of administration and uniformity of dosage. The specific therapeutically or prophylactically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The amount of a compound required to achieve an optimum clinical outcome can vary from subject to subject, depending, for example, on species, age, and general condition of a subject, severity of the side effects, cancer to be treated, identity of the particular compound(s) to be administered, and mode of administration. The desired dosage can be delivered two or three times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day (e.g., once) to a 70 kg adult human may comprise about 1-100 mg, about 1-50 mg, about 1-35 mg (e.g., about 1-5, 1-10, 1-15, 1-20, 1-25, or 1-30 mg), about 2-20 mg, about 3-15 mg or about 10-30 mg (e.g., 10-20 or 10-25 mg). Here, and wherever ranges are referenced, the end points are included. The dosages provided in this disclosure can be scaled for patients of differing weights or body surface and may be expressed per m² of the patient's body surface. In certain embodiments, compositions of the invention may be administered once per day. The dosage of a compound can be about 1-100 mg, about 1-50 mg, about 1-25 mg, about 2-20 mg, about 5-15 mg, about 10-15 mg, or about 13-14 mg. In certain embodiments, a composition of the invention may be administered twice per day. In some embodiments, the dosage of a compound for each administration is about 0.5 mg to about 50 mg, about 0.5 mg to about 25 mg, about 0.5 mg to about 1 mg, about 1 mg to about 10 mg, about 1 mg to about 5 mg, about 3 mg to about 5 mg, or about 4 mg to about 5 mg.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Compound Synthesis

Compounds as shown in Table 1 were synthesized using a method combining solid-phase synthesis with solution phase synthesis according to Scheme 1. These compounds contain 5,6-dihydroimidazo[2,1-b]thiazole (imidazothiazole) and urea motifs with a hydrocarbon linker.

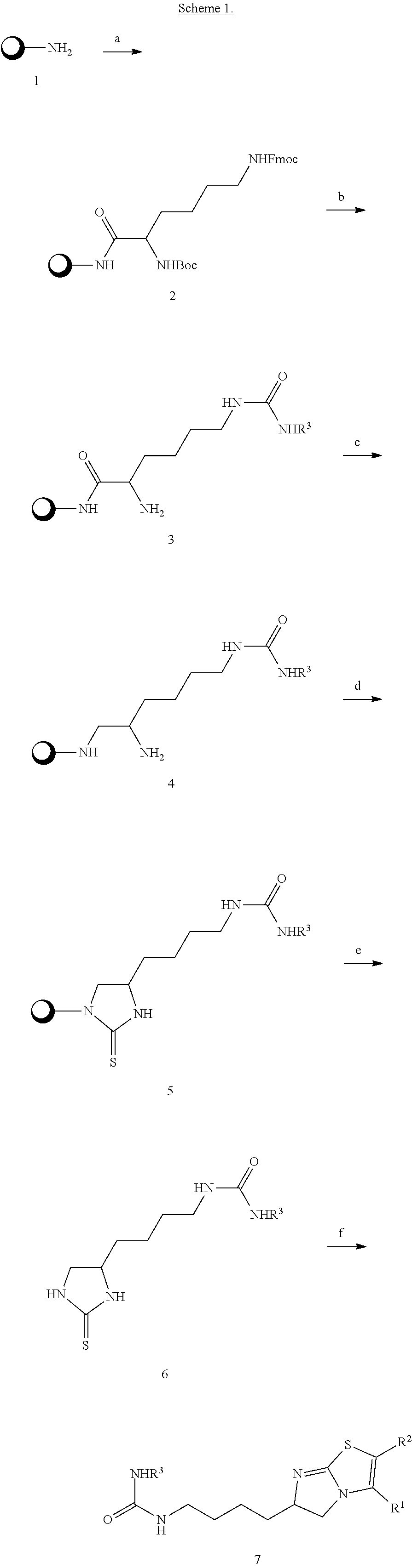

Generally, the solid-phase reaction was carried out using RINK amide resin. A Fmoc protected diamino acid was coupled on resin 1 using standard HBTU/HOBt/DIEA activation to form resin bound 2. The resin bound 2 was then reacted with isocyanate to form resin bound peptide urea 3. Next, the resin bound 3 was reduced using BH₃. THF at 65° C. for 72 hours, followed by treatment with piperidine for 24 hours to form the resin-bound diamine 4. The resin-bound diamine 4 was then reacted with thiocarbonyldiimidazole to form the resin bound intermediate cyclic thiourea 5. The intermediate 6 was then cleaved from the resin with 95% TFA and reacted with a bromoketone to form the final product 7.

For USCYL01, specifically, a Boc-Lys (Fmoc)-OH was coupled on MBHA resin 1 using standard HBTU/HOBt/DIEA activation to form resin bound 2. The resin bound 2 was then treated with 10% piperidine to remove the Fmoc protective group and reacted with phenyl isocyanate to form resin bound peptide urea, followed by treatment with 55% TFA to remove the Boc protective group to form resin bound 3. Next, the resin bound 3 was reduced using BH₃. THF at 65° C. for 72 hours, followed by treatment with piperidine for 24 hours to form the resin-bound diamine 4. The resin-bound diamine 4 was then reacted with 1,1′-thiocarbonyldiimidazole to form the resin bound intermediate cyclic thiourea 5. The intermediate 6 was then cleaved from the resin with 95% TFA and reacted with 2-brom-2′-acetonaphtone to form the product 7.

Trifluoroacetate salts of the compounds were generated by introducing trifluoroacetic acid during reverse phase HPLC purification with TFA being used as a buffer in the mobile phase. The resulting products were USCYL01-USCYL32 as provided in Table 1.

NMR characterization data for USCYL01-USCYL32 (Table 1) are as follows.

### USCYL01

¹H NMR (400 MHZ, DMSO-d6) δ 1.33-1.44 (m, 2H), 1.45-1.53 (m, 2H), 1.76-1.90 (m, 2H), 3.08-3.12 (m, 2H), 4.31-4.39 (m, 1H), 4.74-4.82 (m, 2H), 6.27-6.30 (m, 1H), 6.86 (t, 1H, J=7.2 Hz), 7.18 (t, 2H, J=8 Hz), 7.37 (dd, 2H, J=7.6, 1 Hz), 7.60-7.65 (m, 2H), 7.76 (dd, 2H, J=8.6, 1.8 Hz), 7.99-8.02 (m, 1H), 8.06 (d, 2H, J=8.4 Hz), 8.24 (d, 1H, J=1.2 Hz), 8.51 (s, 1H), 10.25 (br.s, 1H, TFA).

¹³C NMR (100 MHz, DMSO-d6) o 21.9, 29.5, 34.1, 38.7, 52.7, 64.4, 107.6, 116.5 (q, J=290 Hz, TFA), 117.5, 120.9, 124.4, 125.1, 126.7, 127.1, 127.6, 127.7, 128.5, 128.6, 128.9, 132.6, 133.1, 137.3, 140.6, 155.3, 158.1 (q, J=30 Hz, TFA), 170.3.

### USCYL02

¹H NMR (400 MHZ, DMSO-d6) δ 0.98-1.14 (m, 3H), 1.15-1.25 (m, 2H), 1.27-1.43 (m, 4H), 1.44-1.52 (m, 1H), 1.56-1.62 (m, 2H), 1.66-1.72 (m, 2H), 1.74-1.87 (m, 2H), 2.99 (t, 2H, J=6.4 Hz), 3.30 (br.s, 1H), 4.33 (dd, 1H, J=6.4, 8.4 Hz), 4.70-4.81 (m, 2H), 5.70 (d, 2H, J=16 Hz), 7.19 (s, 1H), 7.62 (t, 1H, J=10 Hz), 7.64 (t, 1H, J=10 Hz), 7.76 (dd, 1H, J=8.8, 2 Hz), 7.99-8.03 (m, 1H), 8.05-8.09 (m, 1H), 8.25 (d, 1H, J=0.8 Hz), 10.21 (br.s, 1H)

### USCYL03

¹H NMR (400 MHz, DMSO-d6) δ 1.26-1.43 (m, 2H), 1.45-1.57 (m, 2H), 1.75-1.83 (m, 2H), 2.22 (s, 3H), 3.09 (q, 2H, J=6.4 Hz), 4.31-4.41 (m, 1H), 4.72-4.82 (m, 2H), 6.24 (t, 1H, J=6.4 Hz), 6.69 (d, 1H, J=7.6 Hz), 7.06 (t, 1H, J=7.6 Hz), 7.15 (d, 1H, J=8.4 Hz), 7.19 (s, 1H), 7.21 (s, 1H), 7.60-7.66 (m, 2H), 7.76 (dd, 1H, J=8.8, 2 Hz), 7.99-8.03 (m, 1H), 8.05-8.09 (m, 2H), 8.24 (d, 1H, J=1.2 Hz), 8.40 (s, 1H), 10.22 (br.s, 1H)

### USCYL04

¹H NMR (400 MHZ, DMSO-d6) δ 1.29-1.42 (m, 2H), 1.43-1.50 (m, 2H), 1.72-1.85 (m, 2H), 2.22 (s, 3H), 3.09 (q, 2H, J=6.8 Hz), 3.81 (s, 3H), 4.20 (dd, 1H, J=8, 10 Hz), 4.63 (t, 1H, J=10 Hz), 4.67-4.75 (m, 1H), 6.22 (t, 1H, J=5.6 Hz), 6.69 (d, 1H, J=7.6 Hz), 6.92 (s, 1H), 7.05-7.09 (m, 3H), 7.15-7.17 (m, 1H), 7.21 (s, 1H), 7.58 (dt, 2H, J=6.8, 2 Hz), 8.40 (s, 1H), 10.11 (s, 1H)

### USCYL05

¹H NMR (400 MHZ, DMSO-d6) o 1.37-1.45 (m, 2H), 1.49-1.57 (m, 2H), 1.75-1.89 (m, 2H), 3.17 (q, 2H, J=6.4 Hz), 3.80 (s, 3H), 4.17-4.20 (m, 3H), 4.61 (t, 1H, J=10 Hz), 4.67-4.73 (m, 1H), 5.97 (dd, 1H, J=6 Hz), 6.32 (t, 1H, J=5.6 Hz), 6.93 (s, 1H), 7.07 (dt, 2H, J=8.8, 2.6 Hz), 7.81-7.23 (m, 3H), 7.27-7.31 (m, 2H), 7.59 (dt, 2H, J=8.8, 2.4 Hz), 8.27 (s, 1H), 10.12 (br.s, 1H)

### USCYL06

¹H NMR (400 MHZ, DMSO-d6) δ 1.27-1.36 (m, 2H), 1.38-1.45 (m, 2H), 1.71-1.80 (m, 2H), 3.02 (q, 2H, J=6.0 Hz), 3.81 (s, 3H), 4.21 (dd, 1H, J=8, 10 Hz), 4.63 (t, 1H, J=10 Hz), 4.69-4.77 (m, 1H), 6.64 (t, 1H, J=5.6 Hz), 6.93 (s, 1H), 7.05 (dt, 2H, J=8.8, 2.6 Hz), 7.40 (t, 1H, J=8 Hz), 7.49-7.56 (m, 3H), 7.58 (dt, 2H, J=8.8, 2.4 Hz), 7.88-7.90 (m, 1H), 7.98 (dd, 1H, J=7.6, 0.8 Hz), 8.08 (dd, 1H, J=7.2, 2 Hz), 8.51 (s, 1H), 10.10 (br.s, 1H)

### USCYL07

¹H NMR (400 MHZ, DMSO-d6) δ 1.30-1.40 (m, 2H), 1.42-1.51 (m, 2H), 1.72-1.86 (m, 2H), 3.09 (q, 2H, J=6.4 Hz), 4.22 (dd, 1H, J=8, 10 Hz), 4.63 (t, 1H, J=10 Hz), 4.67-4.75 (m, 1H), 6.25 (t, 1H, J=5.6 Hz), 6.87 (tt, 1H, J=7.6, 1.2 Hz), 7.10 (s, 1H), 7.17-7.22 (m, 2H), 7.35-7.38 (m, 2H), 7.60 (dt, 2H, 8.8, 2.0 Hz), 7.73 (dt, 2H, J=8.8, 2.0 Hz), 8.49 (s, 1H), 10.16 (s, 1H)

### USCYL08

¹H NMR (400 MHZ, DMSO-d6) δ 1.30-1.40 (m, 2H), 1.42-1.51 (m, 2H), 1.72-1.86 (m, 2H), 3.09 (q, 2H, J=6.4 Hz), 4.20 (dd, 1H, J=7.6, 10 Hz), 4.62 (t, 1H, J=10 Hz), 4.67-4.75 (m, 1H), 6.25 (t, 1H, J=5.6 Hz), 6.87 (tt, 1H, J=7.6, 1.2 Hz), 7.04 (s, 1H), 7.17-7.22 (m, 2H), 7.35-7.41 (m, 4H), 7.69-7.74 (m, 2H), 7.73 (dt, 2H, J=8.8, 2.0 Hz), 8.49 (s, 1H), 10.16 (br.s, 1H)

### USCYL09

¹H NMR (400 MHZ, DMSO-d6) δ 0.83 (t, 3H, J=7.2 Hz), 1.17-1.44 (m, 8H), 1.74-1.87 (m, 2H), 2.92 (t, 2H, J=6.8 Hz), 2.99 (t, 2H, J=6.4 Hz), 4.34 (dd, 1H, J=8.8, 6.8 Hz), 4.72-4.82 (m, 2H), 5.78 (br.s, 2H), 7.19 (s, 1H), 7.61-7.66 (m, 2H), 7.76 (dd, 1H, J=8.8, 2 Hz), 7.99-8.03 (m, 1H), 8.05-8.09 (m, 2H), 8.24 (d, 1H, J=1.2 Hz), 10.21 (br.s, 1H)

### USCYL10

¹H NMR (400 MHZ, DMSO-d6) δ 1.35-1.43 (m, 2H), 1.44-1.51 (m, 2H), 1.75-1.83 (m, 2H), 3.09 (q, 2H, J=6.0 Hz), 3.68 (s, 3H), 4.35 (dd, 1H, J=7.6, 5.2 Hz), 4.72-4.82 (m, 2H), 6.12 (t, 1H, J=5.2 Hz), 6.78 (dt, 2H, J=8.8, 3.6 Hz), 7.19 (s, 1H), 7.27 (dt, 2H, J=9.2, 2 Hz), 7.60-7.66 (m, 2H), 7.76 (dd, 1H, J=8.8, 2 Hz), 7.99-8.03 (m, 1H), 8.05-8.09 (m, 2H), 8.24 (d, 1H, J=1.2 Hz), 8.27 (s, 1H), 10.21 (br.s, 1H)

### USCYL11

¹H NMR (400 MHZ, DMSO-d6) δ 0.85 (d, 3H, J=6.8 Hz), 1.2-1.25 (m, 10H), 1.36-1.42 (m, 2H), 1.71-1.79 (m, 2H), 2.91-2.98 (m, 4H), 3.82 (s, 3H), 3.81 (s, 3H), 4.19 (dd, 1H, J=7.6, 10 Hz), 4.61 (t, 1H, J=10 Hz), 4.67-4.79 (m, 1H), 5.78 (br.s, 2H), 6.93 (s, 1H), 7.08 (dt, 2H, J=9.2, 2.6 Hz), 7.59 (dt, 2H, J=8.8, 2.4 Hz), 10.13 (br.s, 1H)

### USCYL12

¹H NMR (400 MHZ, DMSO-d6) δ 1.32-1.40 (m, 2H), 1.43-1.50 (m, 2H), 1.72-1.85 (m, 2H), 2.20 (s, 3H), 3.08 (q, 2H, J=6.8 Hz), 3.81 (s, 3H), 4.19 (dd, 1H, J=8, 10 Hz), 4.62 (t, 1H, J=10 Hz), 4.67-4.79 (m, 1H), 6.20 (t, 1H, J=5.6 Hz), 6.92 (s, 1H), 7.00 (d, 2H, J=8.4 Hz), 7.06 (dt, 2H, J=8.4, 2 Hz), 7.25 (dt, 2H, J=8.4, 2 Hz), 7.58 (dt, 2H, J=8.8, 2 Hz), 8.37 (s, 1H), 10.13 (s, 1H)

### USCYL13

¹H NMR (400 MHZ, DMSO-d6) o 1.36-1.43 (m, 2H), 1.45-1.52 (m, 2H), 1.75-1.88 (m, 2H), 3.10 (q, 2H, J=6.4 Hz), 4.34 (dd, 1H, J=7.6, 8 Hz), 4.72-4.82 (m, 2H), 6.27 (t, 1H, J=5.6 Hz), 7.02 (t, 2H, J=8.8 Hz), 7.18 (s, 1H), 7.37 (dd, 2H, J=4.8, 9.2 Hz), 7.61-7.66 (m, 2H), 7.76 (dd, 1H, J=2, 8.4 Hz), 7.99-8.02 (m, 1H), 8.05-8.08 (m, 2H), 8.24 (br.s, 1H, 1.2 Hz), 8.55 (s, 1H), 10.22 (br.s, 1H)

### USCYL14

¹H NMR (400 MHZ, DMSO-d6) δ 1.31-1.42 (m, 2H), 1.43-1.51 (m, 2H), 1.73-1.83 (m, 2H), 2.16 (s, 3H), 3.10 (q, 2H, J=4 Hz), 3.82 (s, 3H), 4.20 (dd, 1H, J=8, 10 Hz), 4.63 (t, 1H, J=10.4 Hz), 4.67-4.75 (m, 1H), 6.59 (t, 1H, J=5.2 Hz), 6.85 (td, 1H, J=7.2, 0.8 Hz), 6.93 (s, 1H), 7.03-7.11 (m, 4H), 7.57-7.61 (m, 3H), 7.78 (d, 1H, J=7.6 Hz), 10.14 (s, 1H)

### USCYL15

¹H NMR (400 MHZ, DMSO-d6) δ 1.32-1.40 (m, 2H), 1.42-1.50 (m, 2H), 1.72-1.85 (m, 2H), 2.20 (s, 3H), 3.08 (q, 2H, J=6.8 Hz), 3.68 (s, 3H), 3.81 (s, 3H), 4.18 (dd, 1H, J=8, 10 Hz), 4.61 (t, 1H, J=10 Hz), 4.67-4.79 (m, 1H), 6.11 (t, 1H, J=5.6 Hz), 6.79 (dt, 2H, J=9.2, 2.4 Hz), 6.90 (s, 1H), 7.06 (dt, 2H, J=8.4, 2.4 Hz), 7.27 (dt, 2H, J=9.2, 2.4 Hz), 7.58 (dt, 2H, J=8.8, 2.4 Hz), 8.27 (s, 1H), 10.21 (br.s, 1H)

### USCYL16

¹H NMR (400 MHZ, DMSO-d6) δ 1.27-1.36 (m, 2H), 1.38-1.45 (m, 2H), 1.71-1.80 (m, 2H), 3.02 (q, 2H, J=6.0 Hz), 3.81 (s, 3H), 4.21 (m, 3H), 4.63 (t, 1H, J=10 Hz), 4.69-4.77 (m, 1H), 5.97 (t, 1H, J=5.6 Hz), 6.32 (t, 1H, J=5.8 Hz), 6.93 (s, 1H), 7.07 (dt, 2H, J=8.8, 2.6 Hz), 7.18-7.23 (m, 3H), 7.27-7.31 (m, 2H), 7.58 (dt, 2H, J=8.8, 2.4 Hz), 10.10 (br.s, 1H)

### USCYL17

¹H NMR (400 MHZ, DMSO-d6) δ 1.20-1.33 (m, 4H), 1.35-1.40 (m, 2H), 1.42-1.51 (m, 2H), 1.52-1.62 (m, 2H), 1.69-1.81 (m, 4H), 2.98 (q, 2H, J=6.0 Hz), 3.77-3.82 (m, 1H), 3.82 (s, 3H), 4.18 (dd, 1H, J=8, 10 Hz), 4.61 (t, 1H, J=10 Hz), 4.65-4.71 (m, 1H), 5.69 (t, 1H, J=5.6 Hz), 5.77 (d, 1H, J=7.6 Hz), 6.93 (s, 1H), 7.08 (dt, 2H, J=8.8, 2.6 Hz), 7.59 (dt, 2H, J=8.8, 2.4 Hz), 10.11 (br.s, 1H)

### USCYL18

¹H NMR (400 MHZ, DMSO-d6) δ 1.33-1.42 (m, 2H), 1.43-1.51 (m, 2H), 1.73-1.83 (m, 2H), 3.09 (q, 2H, J=6.4 Hz), 3.82 (s, 3H), 4.196 (dd, 1H, J=7.6, 10 Hz), 4.64 (t, 1H, J=10 Hz), 4.67-4.73 (m, 1H), 6.22 (t, 1H, J=5.6 Hz), 6.87 (tt, 1H, J=7.2, 1.2 Hz), 6.92 (s, 1H), 7.06 (dt, 2H, J=8.8, 2.0 Hz), 7.20 (td, 2H, J=7.6, 2 Hz), 7.37 (dt, 2H, J=7.6, 1.2 Hz), 7.58 (dt, 2H, 8.8, 2.4 Hz), 8.47 (s, 1H), 10.08 (s, 1H)

### USCYL19

¹H NMR (400 MHZ, DMSO-d6) δ 0.80 (t, 3H, J=7.2 Hz), 1.15-1.-1.24 (m, 2H), 1.24-1.33 (m, 2H), 2.97 (q, 2H, J=6.4 Hz), 3.31-3.33 (m, 1H), 3.40-3.46 (m, 1H), 4.49 (dd, 1H, J=6.4, 10.0 Hz), 4.70 (t, 1H, J=10 Hz), 4.74-4.81 (m, 1H), 6.07 (t, 1H, J=5.6 Hz), 6.27 (t, 1H, J=6.0 Hz), 7.17 (s, 1H), 7.61-7.66 (m, 2H), 7.74 (dd, 1H, J=8.4, 2 Hz), 7.99-8.06 (m, 2H), 8.08 (d, 1H, J=8.8 Hz), 8.22 (d, 1H, J=0.8 Hz), 10.05 (br.s, 1H)

### USCYL20

¹H NMR (400 MHZ, DMSO-d6) δ 1.33-1.41 (m, 2H), 1.43-1.51 (m, 2H), 1.72-1.86 (m, 2H), 3.09 (q, 2H, J=6.4 Hz), 4.21 (dd, 1H, J=7.6, 9.6 Hz), 4.65 (t, 1H, J=10 Hz), 4.67-4.75 (m, 1H), 6.36 (t, 1H, J=5.6 Hz), 7.10 (s, 1H), 7.23 (dd, 2H, J=2.0, 6.8 Hz), 7.41 (dd, 2H, J=2.0, 6.8 Hz), 7.59 (dd, 2H, J=2, 6.8 Hz), 7.68 (dd, 2H, J=2, 6.8 Hz), 8.71 (s, 1H), 10.18 (br.s, 1H)

### USCYL21

¹H NMR (400 MHZ, DMSO-d6) δ 1.23-1.41 (m, 2H), 1.44-1.51 (m, 2H), 1.71-1.86 (m, 2H), 3.09 (q, 2H, J=6.4 Hz), 4.25 (dd, 1H, J=7.6, 9.6 Hz), 4.65 (t, 1H, J=10 Hz), 4.68-4.76 (m, 1H), 6.33 (t, 1H, J=5.6 Hz), 6.87 (tt, 1H, J=7.6, 1.2 Hz), 7.17-7.22 (m, 2H), 7.25 (s, 1H), 7.37 (dd, 2H, J=8.8, 1.2 Hz), 7.69-7.71 (m, 1H), 8.15 (dd, 1H, J=8.4, 3.6 Hz), 8.55 (s, 1H), 8.71 (dd, 1H, J=2.4, 0.4 Hz)

### USCYL22

¹H NMR (400 MHZ, DMSO-d6) δ 1.33-1.41 (m, 2H), 1.43-1.50 (m, 2H), 1.73-1.84 (m, 2H), 3.09 (q, 2H, J=6.4 Hz), 3.82 (s, 3H), 4.19 (dd, 1H, J=8.0, 10.0 Hz), 4.62 (t, 1H, J=10 Hz), 4.67-4.73 (m, 1H), 6.36 (t, 1H, J=5.6 Hz), 6.92 (s, 1H), 7.02 (dt, 1H, J=2.0, 9.2 Hz), 7.07 (dt, 2H, J=2.8, 8.8 Hz), 7.36-7.41 (m, 2H), 7.58 (dt, 2H, J=8.8, 2.4 Hz), 8.64 (s, 1H), 8.54 (s, 1H), 10.21 (br.s, 1H)

### USCYL23

¹H NMR (400 MHZ, DMSO-d6) δ 1.33-1.42 (m, 2H), 1.43-1.51 (m, 2H), 1.73-1.84 (m, 2H), 3.10 (q, 2H, J=6.4 Hz), 3.82 (s, 3H), 4.19 (dd, 1H, J=8.0, 10.0 Hz), 4.62 (t, 1H, J=10 Hz), 4.67-4.75 (m, 1H), 6.52 (t, 1H, J=5.6 Hz), 6.67 (tdd, 1H, J=0.8, 2.4, 8.8 Hz), 6.92 (s, 1H), 7.02 (ddd, 1H, J=0.4, 1.6, 8.4 Hz), 7.06 (dt, 2H, J=2.8, 8.8 Hz), 7.21 (td, 1H, J=7.2, 8.4 Hz), 7.46 (dt, 1H, J=2.4, 12.4 Hz), 7.58 (dt, 2H, J=2.4, 8.8 Hz), 8.91 (s, 1H), 10.22 (br.s, 1H)

### USCYL24

¹H NMR (400 MHZ, DMSO-d6) δ 1.33-1.41 (m, 2H), 1.44-1.50 (m, 2H), 1.71-1.86 (m, 2H), 2.36 (s, 3H), 3.10 (q, 2H, J=6.4 Hz), 4.19 (dd, 1H, J=8, 10 Hz), 4.62 (t, 1H, J=10 Hz), 4.67-4.74 (m, 1H), 6.30 (t, 1H, J=5.6 Hz), 6.87 (tt, 1H, J=7.2, 1.2 Hz), 6.97 (s, 1H), 7.19 (td, 2H, J=7.6, 1.6 Hz), 7.33 (d, 2H, J=8 Hz), 7.38 (dt, 2H, J=7.6, 1.2), 7.53 (m, 2H), 8.53 (s, 1H), 10.21 (br.s, 1H)

### USCYL25

¹H NMR (400 MHZ, DMSO-d6) δ 1.30-1.42 (m, 2H), 1.44-1.51 (m, 2H), 1.72-1.86 (m, 2H), 3.09 (q, 2H, J=6.4 Hz), 4.20 (dd, 1H, J=7.2, 9.2 Hz), 4.65 (t, 1H, J=10 Hz), 4.68-4.75 (m, 1H), 6.32 (t, 1H, J=5.6 Hz), 6.86 (tt, 1H, J=1.2, 7.2 Hz), 7.16 (s, 1H), 7.18 (dt, 1H, J=2.0, 7.6 Hz) 7.20 (dt, 1H, J=2.0, 7.6 Hz), 7.36-7.39 (m, 2H), 7.46 (d, 1H, J=8 Hz), 7.66 (ddd, 1H, J=1.2, 1.6, 8 Hz), 7.74 (ddd, 1H, J=0.8, 2.0, 8 Hz), 7.87 (t, 1H, J=1.8 Hz), 8.54 (s, 1H), 10.27 (br.s, 1H)

### USCYL26

¹H NMR (400 MHZ, DMSO-d6) o 1.30-1.41 (m, 2H), 1.43-1.51 (m, 2H), 1.72-1.86 (m, 2H), 3.09 (q, 2H, J=6.4 Hz), 4.23 (dd, 1H, J=7.2, 9.6 Hz), 4.65 (t, 1H, J=10 Hz), 4.67-4.75 (m, 1H), 6.30 (t, 1H, J=5.6 Hz), 6.87 (tt, 1H, J=1.2, 7.2 Hz), 7.20 (s, 1H), 7.18 (dt, 1H, J=2.0, 7.6 Hz) 7.20 (dt, 1H, J=2.0, 7.6 Hz), 7.36-7.39 (m, 2H), 7.64 (dd, 1H, J=2, 8.4 Hz), 7.79 (d, 1H, J=8.4 Hz), 7.95 (d, 1H, J=2.4 Hz), 8.52 (s, 1H), 10.25 (br.s, 1H)

### USCYL27

¹H NMR (400 MHZ, DMSO-d6) δ 1.24-1.38 (m, 2H), 1.40-1.47 (m, 2H), 1.66-1.85 (m, 2H), 3.06 (2H, t, J=6.4 Hz), 3.75 (dd, 1H, J=8, 10.4 Hz), 4.20 (t, 1H, J=10.4 Hz), 4.70 (m, 1H), 6.43 (t, 1H, J=5.2 Hz), 6.66 (td, 1H, J=2, 8.4 Hz), 7.01 (dd, 1H, J=1.2, 8.4 Hz), 7.06 (s, 1H), 7.21 (dd, 1H, J=8, 15.2 Hz), 7.44 (dt, 1H, J=2, 12.4 Hz), 7.63-7.66 (m, 3H), 7.70 (dd, 1H, J=1.2, 7.2 Hz), 7.96-7.99 (m, 1H), 8.06-8.08 (m, 1H), 8.14 (d, 1H, J=8.0 Hz), 8.84 (s, 1H), 10.25 (br.s, 1H)

### USCYL28

¹H NMR (400 MHZ, DMSO-d6) o 1.30-1.41 (m, 2H), 1.44-1.51 (m, 2H), 1.72-1.87 (m, 2H), 3.08 (t, 1H, J=8 Hz), 3.10 (t, 1H, J=8 Hz), 4.22 (dd, 1H, J=7.6, 9.6 Hz), 4.62-4.76 (m, 2H), 6.31 (t, 1H, J=5.6 Hz), 6.87 (tt, 1H, J=7.2, 1.2 Hz), 7.05 (s, 1H), 7.17-7.21 (m, 2H), 7.38 (dt, 2H, J=8.4, 1.2 Hz), 7.51-7.55 (m, 3H), 7.64-7.66 (m, 2H), 8.53 (s, 1H), 10.54 (br.s, 1H)

### USCYL29

¹H NMR (400 MHZ, DMSO-d6) δ 1.91-2.04 (m, 2H), 3.24-3.29 (m, 2H), 4.36-4.43 (m, 1H), 4.78-4.80 (m, 2H), 6.51 (t, 1H, J=6.0 Hz), 6.89 (tt, 1H, J=7.6, 0.8 Hz), 7.17 (s, 1H), 7.19-7.23 (m, 2H), 7.39 (dt, 2H, J=7.6, 0.8 Hz), 7.61-7.64 (m, 2H), 7.74 (dd, 1H, J=8.8, 2 Hz), 8.00-8.03 (m, 2H), 8.07 (d, 1H, J=8.8 Hz), 8.218 (d, 1H, J=1.2 Hz), 8.73 (s, 1H), 10.6 (br.s, 1H)

### USCYL30

¹H NMR (400 MHZ, DMSO-d6) δ 1.49-1.61 (m, 2H), 1.78-1.86 (m, 2H), 3.14 (q, 2H, J=8 Hz), 4.35 (dd, 1H, J=12, 20 Hz), 4.79-4.80 (m, 2H), 6.27 (t, 1H, J=6.0 Hz), 6.87 (tt, 1H, J=7.2, 1.2 Hz), 7.18-7.22 (m, 3H), 7.38 (dt, 2H, J=7.6, 1.2 Hz), 7.62-7.64 (m, 2H), 7.76 (dd, 1H, J=8.8, 2 Hz), 7.99-8.02 (m, 1H), 8.05-8.08 (m, 2H), 8.241 (d, 1H, J=1.2 Hz), 8.49 (s, 1H), 10.15 (br.s, 1H)

### USCYL31

¹H NMR (400 MHZ, DMSO-d6) δ 0.97 (d, 6H, J=6.4 Hz), 1.27-1.43 (m, 4H), 1.75-1.85 (m, 2H), 2.98 (t, 2H, J=6.4 Hz), 3.56-3.64 (m, 1H), 4.33 (dd, 1H, J=8.4, 6.4 Hz), 4.70-4.81 (m, 2H), 5.60 (br.s, 1H), 5.71 (br.s, 1H), 7.19 (s, 1H), 7.60-7.66 (m, 2H), 7.76 (dd, 1H, J=8.4, 2 Hz), 7.99-8.03 (m, 1H), 8.05-8.09 (m, 2H), 8.24 (d, 1H, J=1.2 Hz), 10.21 (br.s, 1H)

### USCYL32

¹H NMR (400 MHz, DMSO-d6) δ 0.82 (t, 3H, J=7.2 Hz), 1.80-1.24 (m, 2H), 1.24-1.36 (m, 2H), 2.98 (q, 2H, J=6.4 Hz), 3.27-3.31 (m, 1H), 3.40-3.46 (m, 1H), 4.10 (dd, 1H, J=6.4, 10.4 Hz), 4.34 (t, 1H, J=6.4 Hz), 4.65-4.71 (m, 1H), 6.07 (t, 1H, J=5.6 Hz), 6.27 (t, 1H, J=6.0 Hz), 7.17-7.21 (m, 2H), 7.33-7.36 (m, 3H), 7.43-7.46 (m, 2H), 7.48-7.54 (m, 3H), 10.37 (br.s, 1H)

### IDO1 and TDO Inhibition

Compounds USCYL01-USCYL32 were tested for activity in inhibiting IDO1 and TDO.

A HeLa cell-based assay was used to test for inhibiting IDO1. In a black 96-well plate, 10 μL of tested samples were added. HeLa cells were then added at a density of 10,000 cells/well in 90 μL of complete medium with 0.3 mM Trp and 100 ng/ml IFN-γ. The cells were incubated at 37° C. with 5% CO₂. After 48 hours incubation, 24 μL of 0.5 M methyl isonipecotate were added and incubated at 37° C. without CO₂ overnight. Fluorescence was measured using an iD5 plate reader (Molecular Device) (excitation 400/25 nm; emission 510/20 nm). Results are shown in Table 1.

A SW-48 cell-based assay was used to test for inhibiting TDO. In a black 96-well plate, 10 μL of tested samples were added. SW48 cells were then added at a density of 20,000 cells/well in 90 μL of complete medium containing 0.3 mM Trp. The cells were incubated at 37° C. with 5% CO₂. After 48 hours incubation, 24 μL of 0.5 M methyl isonipecotate were added and incubated at 37° C. without CO₂ overnight. Fluorescence was measured using an iD5 plate reader (Molecular Device) (excitation 400/25 nm; emission 510/20 nm).

Results are shown in Table 1. Compounds having p-methoxyphenyl, p-chlorophenyl, p-bromophenyl, 2-nathphyl groups at the 3-position of the imidazothiazole (R¹), a 4-carbon linker (L), and phenyl- or substituted phenyl (R³) urea were the most potent against both IDO1 and TDO.

TABLE 1
Inhibition of IDO1 and TDO with USCYL01-USCYL32.*

| Compound | | | | | EC50/μM | EC50/μM |
|---|---|---|---|---|---|---|
| ID | R¹ | R² | R³ | L | IDO1 | TDO |
| USCYL01 | 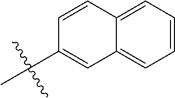 [SMILES: *c1ccc2ccccc2c1] | H | 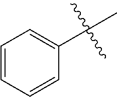 [SMILES: *c1ccccc1] | —(CH₂)₄— | 5.3 | 10.1 |
| USCYL02 | 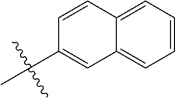 [SMILES: *c1ccc2ccccc2c1] | H | 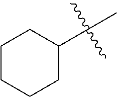 [SMILES: *C1CCCCC1] | —(CH₂)₄— | 7.4 | 17.0 |
| USCYL03 | 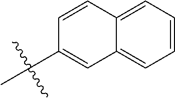 [SMILES: *c1ccc2ccccc2c1] | H | 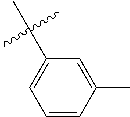 [SMILES: *c1cccc(C)c1] | —(CH₂)₄— | 6.6 | 13.5 |
| USCYL04 | 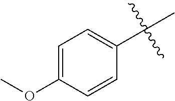 [SMILES: *c1ccc(OC)cc1] | H | 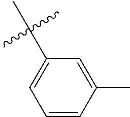 [SMILES: *c1cccc(C)c1] | —(CH₂)₄— | 3.1 | 9.7 |
| USCYL05 | 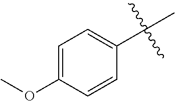 [SMILES: *c1ccc(OC)cc1] | H | 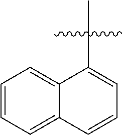 [SMILES: *c1cccc2ccccc12] | —(CH₂)₄— | 6.2 | 9.4 |
| USCYL06 | 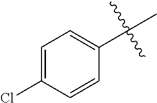 [SMILES: *c1ccc(Cl)cc1] | H | 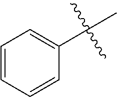 [SMILES: *c1ccccc1] | —(CH₂)₄— | 2.3 | 12.2 |
| USCYL07 | 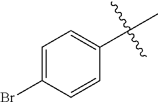 [SMILES: *c1ccc(Br)cc1] | H | 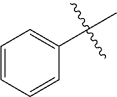 [SMILES: *c1ccccc1] | —(CH₂)₄— | 2.4 | 12.7 |
| USCYL08 | 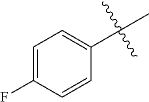 [SMILES: *c1ccc(F)cc1] | H | 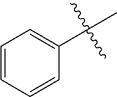 [SMILES: *c1ccccc1] | —(CH₂)₄— | 4.3 | 22.4 |
| USCYL09 | 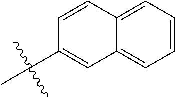 [SMILES: *c1ccc2ccccc2c1] | H | 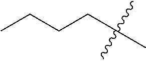 [SMILES: *CCCC] | —(CH₂)₄— | 7.9 | 25.5 |
| USCYL10 | 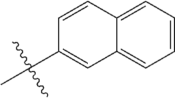 [SMILES: *c1ccc2ccccc2c1] | H | 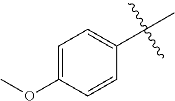 [SMILES: *c1ccc(OC)cc1] | —(CH₂)₄— | 5.9 | 20.3 |
| USCYL11 | 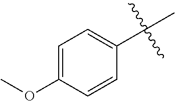 [SMILES: *c1ccc(OC)cc1] | H | 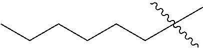 [SMILES: *CCCCCC] | —(CH₂)₄— | 6.9 | 11.1 |
| USCYL12 | 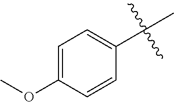 [SMILES: *c1ccc(OC)cc1] | H | 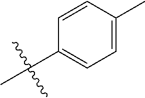 [SMILES: *c1ccc(C)cc1] | —(CH₂)₄— | 1.3 | 32.1 |
| USCYL13 | 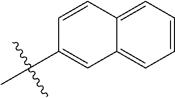 [SMILES: *c1ccc2ccccc2c1] | H | 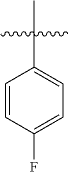 [SMILES: *c1ccc(F)cc1] | —(CH₂)₄— | 7.0 | 15.5 |
| USCYL14 | 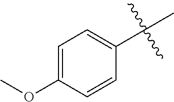 [SMILES: *c1ccc(OC)cc1] | H | 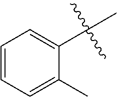 [SMILES: *c1ccccc1C] | —(CH₂)₄— | 4.8 | 23.6 |
| USCYL15 | 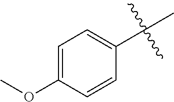 [SMILES: *c1ccc(OC)cc1] | H | 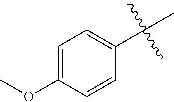 [SMILES: *c1ccc(OC)cc1] | —(CH₂)₄— | 4.4 | 34.5 |
| USCYL16 | 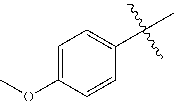 [SMILES: *c1ccc(OC)cc1] | H | 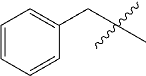 [SMILES: *Cc1ccccc1] | —(CH₂)₄— | 15.7 | 30.3 |
| USCYL17 | 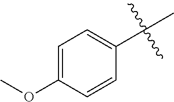 [SMILES: *c1ccc(OC)cc1] | H | 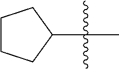 [SMILES: *C1CCCC1] | —(CH₂)₄— | 5.5 | 57.9 |
| USCYL18 | 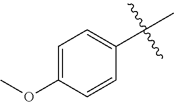 [SMILES: *c1ccc(OC)cc1] | H | 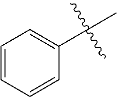 [SMILES: *c1ccccc1] | —(CH₂)₄— | 1.22 | 20.3 |
| USCYL19 | 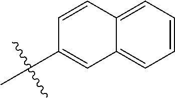 [SMILES: *c1ccc2ccccc2c1] | H | 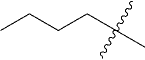 [SMILES: *CCCC] | —CH₂— | 14.0 | 32.3 |
| USCYL20 | 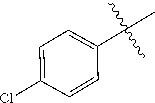 [SMILES: *c1ccc(Cl)cc1] | H | 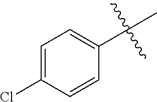 [SMILES: *c1ccc(Cl)cc1] | —(CH₂)₄— | 2.7 | 12.7 |
| USCYL21 | 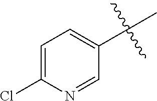 [SMILES: *c1ccc(Cl)nc1] | H | 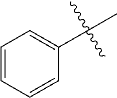 [SMILES: *c1ccccc1] | —(CH₂)₄— | 5.9 | 100 |
| USCYL22 | 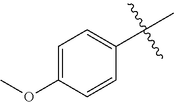 [SMILES: *c1ccc(OC)cc1] | H | 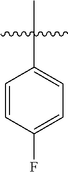 [SMILES: *c1ccc(F)cc1] | —(CH₂)₄— | 0.92 | 100 |
| USCYL23 | 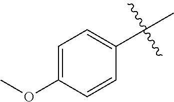 [SMILES: *c1ccc(OC)cc1] | H | 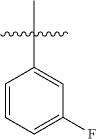 [SMILES: *c1cccc(F)c1] | —(CH₂)₄— | 1.10 | 25.8 |
| USCYL24 | 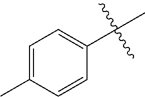 [SMILES: *c1ccc(C)cc1] | H | 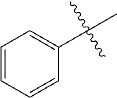 [SMILES: *c1ccccc1] | —(CH₂)₄— | 1.90 | 15.1 |
| USCYL25 | 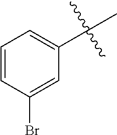 [SMILES: *c1cccc(Br)c1] | H | 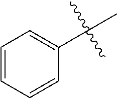 [SMILES: *c1ccccc1] | —(CH₂)₄— | 1.88 | 11.8 |
| USCYL26 | 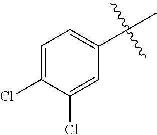 [SMILES: *c1ccc(Cl)c(Cl)c1] | H | 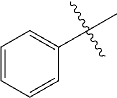 [SMILES: *c1ccccc1] | —(CH₂)₄— | 3.50 | 10.6 |
| USCYL27 | 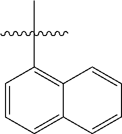 [SMILES: *c1cccc2ccccc12] | H | 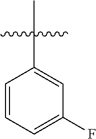 [SMILES: *c1cccc(F)c1] | —(CH₂)₄— | 5.13 | 8.78 |
| USCYL28 | 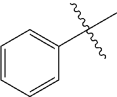 [SMILES: *c1ccccc1] | H | 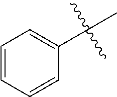 [SMILES: *c1ccccc1] | —(CH₂)₄— | 3.41 | 100 |
| USCYL29 | 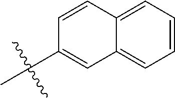 [SMILES: *c1ccc2ccccc2c1] | H | 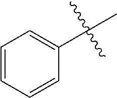 [SMILES: *c1ccccc1] | —(CH₂)₂— | 4.0 | 19.2 |
| USCYL30 | 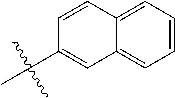 [SMILES: *c1ccc2ccccc2c1] | H | 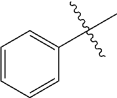 [SMILES: *c1ccccc1] | —(CH₂)₃— | 5.46 | 13.9 |
| USCYL31 | 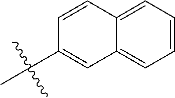 [SMILES: *c1ccc2ccccc2c1] | H | 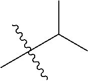 [SMILES: *C(C)C] | —(CH₂)₄— | 28.1 | 100 |
| USCYL32 | 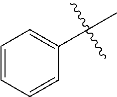 [SMILES: *c1ccccc1] | 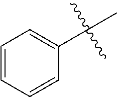 [SMILES: *c1ccccc1] | 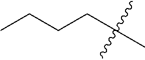 [SMILES: *CCCC] | —CH₂— | 26.8 | 100 |

*Compounds USCYL01-USCYL32 are trifluoroacetate salts of compounds of Formula (I):

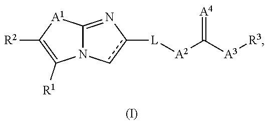

wherein A¹ is S, A² and A³ are each NH, A⁴ is O; - - - is absent, and R¹, R², R³, and L are as defined in Table 1.

### Cancer Treatment

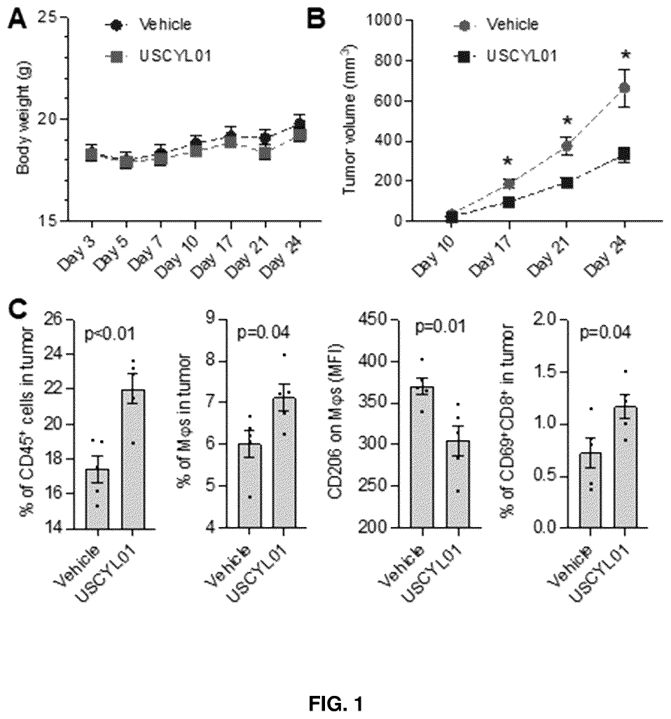

In a mouse model of breast cancer, 8-week-old female C57Bl/6 mice were inoculated with EO771 cells in the 4th pair of mammary fat pads. The next day they started to receive i.p. USCYL01 (10 mg/kg) or vehicle control every other day for 3 weeks (total 10 doses/mouse) (10 mice/group). Mice were sacrificed 24 days after cell inoculation. USCYL01 treatment did not affect body weight, and the weights of the heart, liver, lungs, kidney, brain, and lymph nodes, but it significantly suppressed tumor growth (FIG. 1 (A and B)). Flow cytometry analysis of immune cells in the tumors (5 tumors/group) shows that total leukocyte (CD45+) percentage, macrophages (MΦ) (CD68+) percentage, and activated CD8+T cell (CD69+CD8+) percentage were increased in tumors of the mice treated with USCYL01 (FIG. 1 (C)), and that USCYL01 treatment reduced CD206 expression in the infiltrating Mos in the tumors (FIG. 1 (C)), indicating that USCYL01 treatment suppressed breast tumor growth by improving anti-tumor immune response.

### Analgesic Effect

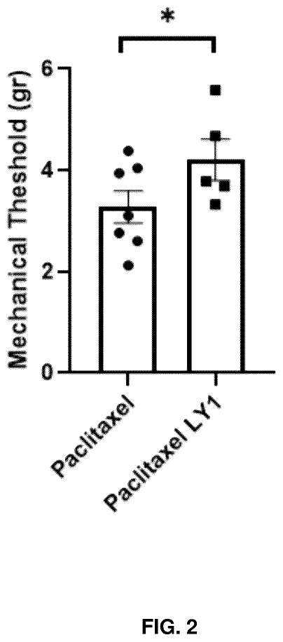

In a mouse model of paclitaxel-induced neuropathic pain, USCYL01 promoted significant analgesic effect in male mice (FIG. 2).

What is claimed is:

1. A compound of Formula (I): 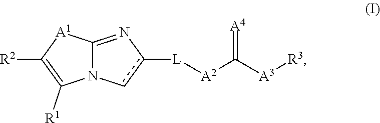
or a salt thereof, wherein:
A¹, A², A³, and A⁴ are each independently O, S, or NR⁴;
- - - represents a bond that is present or absent;
L is a bond or optionally substituted alkylene optionally containing one or more heteroatom(s);
R¹, R², R³, and R⁴ in each instance is independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl, with the proviso that R³ and at least one of R¹ and R² are not hydrogen.

2. The compound of claim 1, wherein R¹ is Formula (II), Formula (III), Formula (IV), Formula (V), Formula (VI), Formula (VII), Formula (VIII), Formula (IX), or Formula (X): 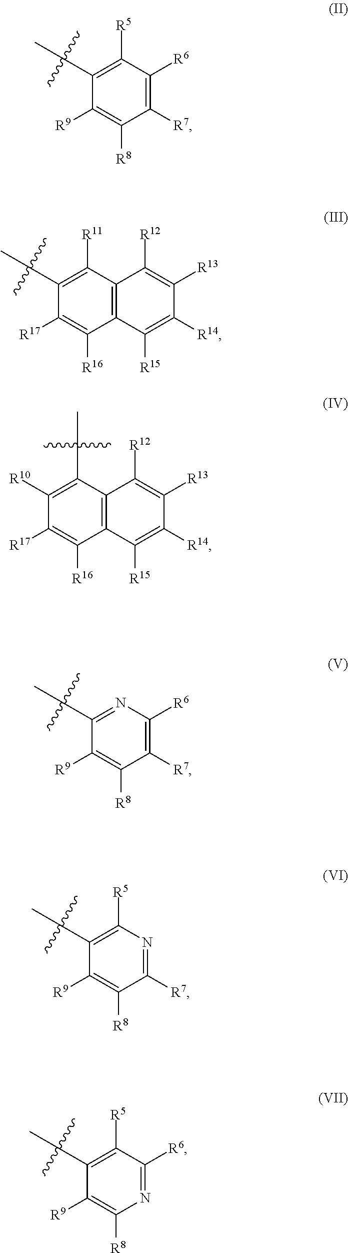 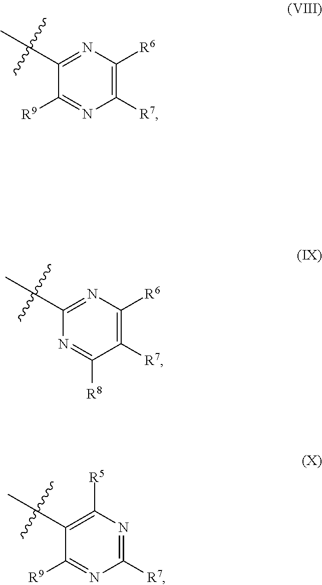
wherein R⁵—R¹⁷ of R¹ are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

3. The compound of claim 2, wherein R¹ is:
any one of Formulas II, V, VI, VII, VIII, IX, and X wherein at least one of R⁶—R⁸ is not hydrogen; or
any one of Formulas III and IV.

4. The compound of claim 2, wherein R¹ is:
any one of Formulas II, V, VI, VII, VIII, IX, and X wherein at least one of R⁶—R⁸ is halogen, optionally substituted alkyloxy, or optionally substituted alkyl; or
any one of Formulas III and IV.

5. The compound of claim 2, wherein R¹ is:
any one of Formulas II, V, VI, VII, VIII, IX, and X wherein at least one of R⁶—R⁸ is halogen or optionally substituted alkyloxy; or
any one of Formulas III and IV.

6. The compound of claim 2, wherein R³ is optionally substituted alkyl, optionally substituted cycloalkyl, Formula (II), Formula (III), Formula (IV), Formula (V), Formula (VI), Formula (VII), Formula (VIII), Formula (IX), or Formula (X), wherein R⁵—R¹⁷ of R³ are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

7. The compound of claim 6, wherein R³ is Formula (II), Formula (III), Formula (IV), Formula (V), Formula (VI), Formula (VII), Formula (VIII), Formula (IX), or Formula (X).

8. The compound of claim 6, wherein R³ is Formula II, Formula III, or Formula IV.

9. The compound of claim 6, wherein R³ is Formula II, wherein at least one of R⁶—R⁸ is halogen or optionally substituted alkyl.

10. The compound of claim 6, wherein R³ is Formula II, wherein at least one of R⁶—R⁸ is halogen.

11. The compound of claim 6, wherein:
A¹ is S;
A² is NR⁴;
A³ is NR⁴;
A⁴ is O;
- - - is absent; and
L is optionally substituted C1-C6 alkylene.

12. The compound of claim 1, wherein R³ is optionally substituted alkyl, optionally substituted cycloalkyl, Formula (II), Formula (III), Formula (IV), Formula (V), Formula (VI), Formula (VII), Formula (VIII), Formula (IX), or Formula (X): 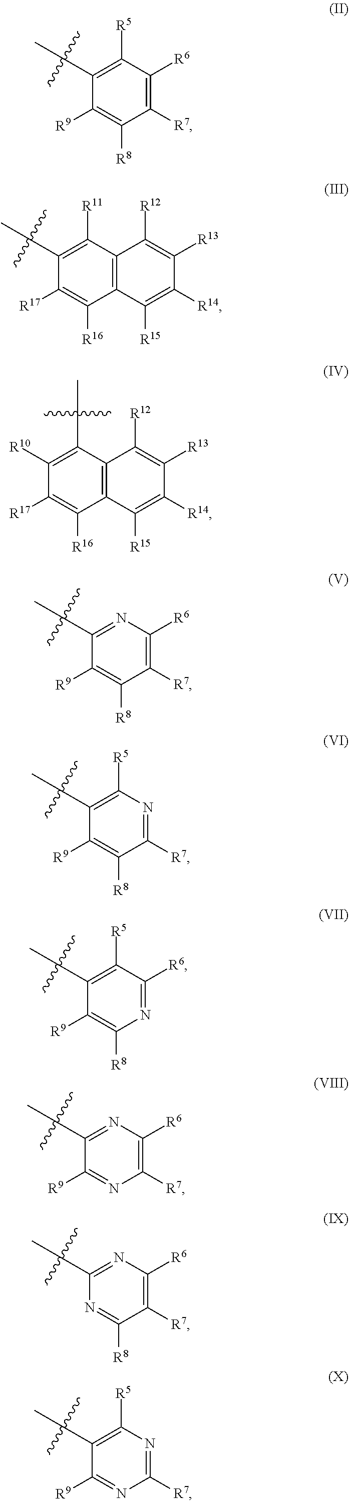
wherein R⁵—R¹⁷ of R³ are each independently hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, sulfino, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclyl.

13. The compound of claim 12, wherein R³ is Formula (II), Formula (III), Formula (IV), Formula (V), Formula (VI), Formula (VII), Formula (VIII), Formula (IX), or Formula (X).

14. The compound of claim 12, wherein R³ is Formula II, Formula III, or Formula IV.

15. The compound of claim 12, wherein R³ is Formula II, wherein at least one of R⁶—R⁸ is halogen or optionally substituted alkyl.

16. The compound of claim 12, wherein R³ is Formula II, wherein at least one of R⁶—R⁸ is halogen.

17. The compound of claim 1, wherein the compound is any one of the following, or a salt and/or enantiomer thereof:

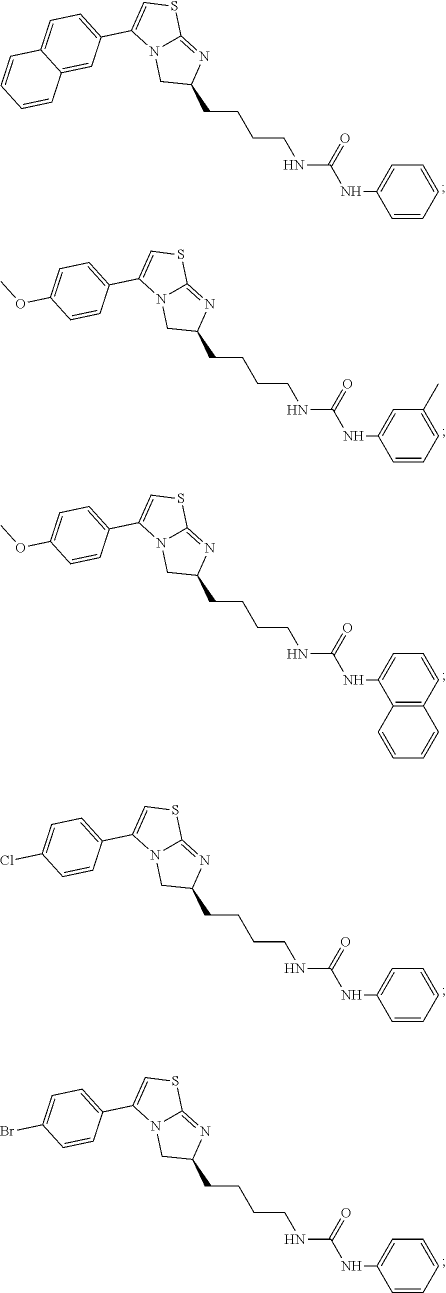

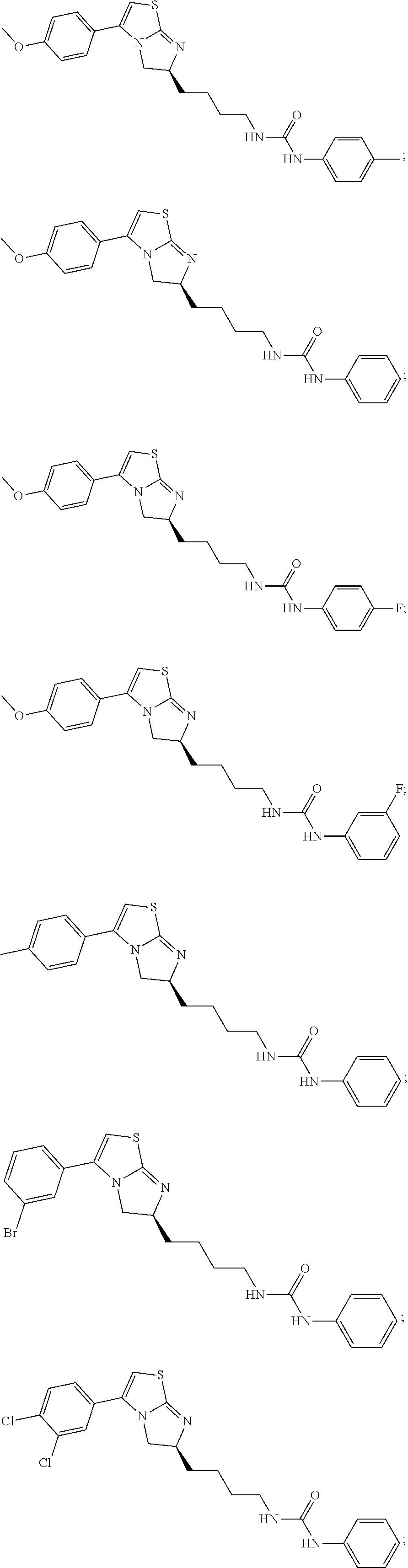

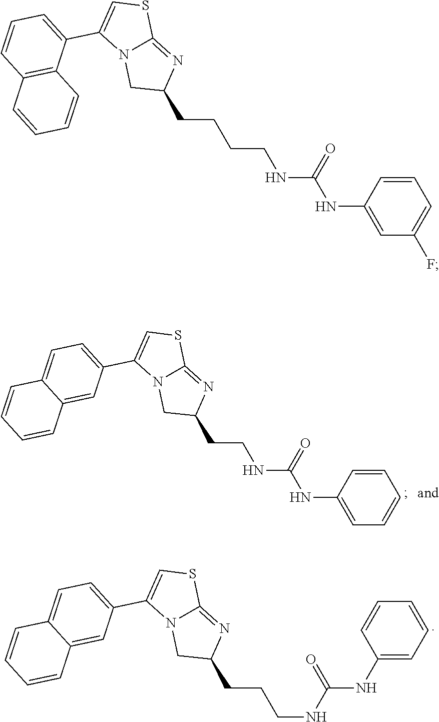 [SMILES: O=C(NCCCC[C@H]1CN2C(c3cccc4ccccc34)=CSC2=N1)Nc1cccc(F)c1 ; O=C(NCC[C@H]1CN2C(c3ccc4ccccc4c3)=CSC2=N1)Nc1ccccc1 ; O=C(NCCC[C@H]1CN2C(c3ccc4ccccc4c3)=CSC2=N1)Nc1ccccc1]

18. A method of inhibiting indoleamine 2,3-dioxygenase 1 (IDO1) and/or tryptophan 2,3-dioxygenase (TDO), the method comprising contacting the IDO1 and/or the TDO with the compound of claim 1 in an amount effective to inhibit the IDO1 and/or the TDO.

19. A method of treating a condition mediated by indoleamine 2,3-dioxygenase 1 (IDO1) and/or tryptophan 2,3-dioxygenase (TDO) in a subject, the method comprising administering the compound of claim 1 to the subject in an amount effective to treat the condition.

20. The method of claim 19, wherein the condition is cancer or neuropathic pain.
